(19) United States
(12) Patent Application Publication
(10) Pub. No.: US 2026/0256438 A1
(43) Pub. Date: Sep. 3, 2026

(54) RADIATION THERAPY SYSTEM WITH GANTRY-MOUNTED X-RAY IMAGING SYSTEM AND SUPPLEMENTAL X-RAY IMAGING SYSTEM

(71) Applicant: SIEMENS HEALTHINEERS INTERNATIONAL AG, Steinhausen (CH)

(72) Inventors: Pascal PAYSAN, Basel (CH); Reto FILIBERTI, Baar (CH); Daniel MORF, Buch am Irchel (CH); Maite RUIZ YANIZ, München (DE)

(73) Assignee: SIEMENS HEALTHINEERS INTERNATIONAL AG, Steinhausen (CH)

(21) Appl. No.: 19/069,164

(22) Filed: Mar. 3, 2025

(51) Int. Cl. A61B 6/00; A61B 6/40; G06T 7/00

(52) U.S. Cl. CPC A61B 6/4447; A61B 6/4014; G06T 7/00; G06T 2207/10116

(57) ABSTRACT

A radiation treatment system includes a rotatable gantry operable to rotate about a treatment isocenter of the radiation therapy system, a treatment radiation source mounted on the rotatable gantry, a first x-ray imaging system mounted on the rotatable gantry, a second x-ray imaging system that is fixed in position while the rotatable gantry rotates about the treatment isocenter, and a controller. The controller performs the operations of causing the first x-ray imaging system to generate first image information for a region surrounding the treatment isocenter, causing the second x-ray imaging system to generate second image information for the region surrounding the treatment isocenter, and receiving the first image information from the first x-ray imaging system and the second image information from the second x-ray imaging system.

### BACKGROUND

Unless otherwise indicated herein, the approaches described in this section are not prior art to the claims in this application and are not admitted to be prior art by inclusion in this section.

Radiation therapy (also called radiotherapy) is a cancer treatment that employs high doses of ionizing radiation, such as X-rays or high-energy electrons, protons, or other heavy charged particles, to kill cancer cells. Generally, radiation therapy is a localized treatment for a specific target tissue, such as a cancerous tumor. Ideally, radiation therapy is performed on a planning target volume (i.e., the target tissue) that spares the surrounding normal tissue from receiving doses above specified tolerances, thereby minimizing risk of damage to healthy tissue. For example, to accurately supply a planned radiation dose, the spatial distribution of delivered radiation dose within the patient must closely match the spatial distribution of the planned radiation dose. So that the planned radiation dose is correctly supplied to the planning target volume during radiation therapy, the patient should be correctly positioned relative to the radiation source that provides the radiation therapy. In addition, precisely controlling the position of the radiation source relative to the patient is a significant factor in accurately targeting tissue in the patient. In light of the above, to detect and/or compensate for patient motion during a particular radiation therapy session, or “fraction,” patient motion is often monitored in near-real time using optical and/or X-ray imaging techniques.

### SUMMARY

According to various embodiments, a radiation therapy system with a rotatable gantry includes a gantry-mounted X-ray imaging system and a supplemental X-ray imaging system, where the supplemental X-ray imaging system is fixed in position while the rotatable gantry rotates about a treatment isocenter. In some embodiments, an X-ray detector and an X-ray source of the supplemental X-ray imaging system are mounted on a treatment couch of the radiation therapy system. Alternatively, in some embodiments, an X-ray detector and/or an X-ray source of the supplemental X-ray imaging system are mounted on a surface external to the rotatable gantry or couch of the radiation therapy system, such as a floor of a treatment room containing the radiation therapy system or a ceiling of the treatment room containing the radiation therapy system. Control of the supplemental X-ray imaging system is integrated into the radiation therapy system. Therefore, the supplemental X-ray imaging system can be employed synchronously with the gantry-mounted X-ray imaging as a stereo kilovolt imaging chain that delivers three-dimensional information about patient position and internal structures.

According to some embodiments, a radiation treatment system includes: a rotatable gantry operable to rotate about a treatment isocenter of the radiation therapy system; a treatment radiation source mounted on the rotatable gantry; a first x-ray imaging system mounted on the rotatable gantry; a second x-ray imaging system that is fixed in position while the rotatable gantry rotates about the treatment isocenter; and a controller. The controller performs the steps of causing the first x-ray imaging system to generate first image information for a region surrounding the treatment isocenter; causing the second x-ray imaging system to generate second image information for the region surrounding the treatment isocenter; and receiving the first image information from the first x-ray imaging system and the second image information from the second x-ray imaging system.

According to some embodiments, for a radiation therapy system that includes a rotatable gantry operable to rotate about a treatment isocenter of the radiation therapy system, a method of imaging a region of patient anatomy includes: causing a first x-ray imaging system mounted on the rotatable gantry to generate first image information for the region of patient anatomy; causing a second x-ray imaging system of the radiation therapy system to generate second image information for the region of patient anatomy, wherein the second x-ray imaging system is fixed in position while the rotatable gantry rotates about the treatment isocenter; receiving the first image information from the first x-ray imaging system and the second image information from the second x-ray imaging system; and generating a first image of the region of patient anatomy based on the first image information and a second image of the region of patient anatomy based on the second image information.

Further embodiments include a non-transitory computer-readable storage medium comprising instructions that cause a computer system to carry out the above method, as well as a computer system configured to carry out the above method.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. These drawings depict only several embodiments in accordance with the disclosure and are, therefore, not to be considered limiting of its scope. The disclosure will be described with additional specificity and detail through use of the accompanying drawings.

FIG. 1 is a perspective view of a radiation therapy system, according to various embodiments.

FIG. 2 schematically illustrates a side view of the radiation therapy system of FIG. 1, according to various embodiments.

FIG. 3 is a conceptual side view of a couch-positioning assembly and a supplemental X-ray imaging system, in which an imaging panel of the supplemental X-ray imaging system is mounted on the couch-positioning assembly, according to various embodiments.

FIG. 4 schematically illustrates a plan view of a treatment couch, a couch-positioning assembly, and a supplemental X-ray imaging system, according to various embodiments.

FIG. 5 is a conceptual side view of a couch-positioning assembly and a supplemental X-ray imaging system, in which an imaging panel of the supplemental X-ray imaging system is mounted on a treatment couch, according to various embodiments.

FIG. 6 is a conceptual side view of a couch-positioning assembly and a supplemental X-ray imaging system, in which an imaging panel of the supplemental X-ray imaging system is mounted on a surface of a floor or a surface of a turntable, according to various embodiments.

FIG. 7 is a conceptual side view of a couch-positioning assembly and a supplemental X-ray imaging system, in which an X-ray source of the supplemental X-ray imaging system is mounted on a treatment couch, according to various embodiments.

FIG. 8 is a perspective view of another radiation therapy system, according to various embodiments.

FIG. 9 schematically illustrates a base stand and a gantry of the radiation therapy system of FIG. 8, according to various embodiments.

FIG. 10 is a flowchart illustrating the steps of a computer-implemented process for marker tracking in a radiation therapy system, according to various embodiments.

FIG. 11 is a flowchart illustrating the steps of a computer-implemented process for 2D-3D matching in a radiation therapy system, according to various embodiments.

FIG. 12 is a flowchart illustrating the steps of a computer-implemented process for patient model matching in a radiation therapy system, according to various embodiments.

FIG. 13 is an illustration of a computing device configured to perform various embodiments of the present disclosure.

FIG. 14 is a block diagram of an illustrative embodiment of a computer program product for implementing a method for imaging a region of patient anatomy for a radiation therapy system that includes a rotatable gantry operable to rotate about a treatment isocenter of the radiation therapy system, according to various embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the disclosure, as generally described herein, and illustrated in the figures, can be arranged, substituted, combined, and designed in a wide variety of different configurations, all of which are explicitly contemplated and make part of this disclosure. Although the terms “first” and “second” are used to describe various elements, these elements should not be limited by these terms. These terms are used to distinguish one element from another. For example, a first element may be referred to as a second element, and vice versa. Independent of the grammatical term usage, individuals with male, female or other gender identities are included within the term.

### Introduction

In radiation therapy, a patient should be correctly positioned relative to the linear accelerator that provides the radiation therapy so that the prescribed dose is correctly supplied to the planning target volume (i.e., the target tissue). Typically, dosimetric and geometric data are checked before and during the treatment, to ensure correct patient placement and that the administered radiotherapy treatment matches the previously planned treatment. This process is referred to as image guided radiation therapy (IGRT), and involves the use of an imaging system to view target tissues immediately before radiation treatment is delivered to the planning target volume. In many instances, as part of IGRT, to detect and/or compensate for patient motion during a particular radiation therapy session, or “fraction,” patient motion is monitored in real time or near-real time using optical and/or X-ray imaging techniques.

Various surrogate monitoring technologies have been developed to infer intra-fraction motion of a patient and/or internal anatomy of the patient, for example by optically monitoring a surface of the body of the patient. However, optical monitoring technologies cannot directly monitor internal anatomy, where significant motion oftentimes occurs (e.g., due to sporadic prostate movement, variation in bowel or bladder content, and the like). Because there can oftentimes be no reliable correlation between internal anatomy motion and the surface being monitored, optical surface monitoring is frequently unable to detect certain internal anatomy motion, making such monitoring less suitable for detecting intra-fraction motion. Consequently, IGRT typically relies on X-ray imaging of the internal anatomy of a patient to detect intra-fraction motion of the patient and/or internal anatomy of the patient.

IGRT incorporates X-ray imaging coordinates from a treatment plan to ensure the patient is properly aligned for treatment in the radiation therapy device, thereby enabling an increase in accuracy and precision of treatment delivery, shorter treatment times (e.g., hypo-fractionated therapy and/or flash therapy), and patient-individualized therapy. Using IGRT, an adaptive therapy workflow with plan adaptation (“plan of the day”) can be performed, which is based on the actual patient anatomy on the day of treatment. For instance, during a course of radiotherapy, which can take place over many days, the planning target volume and/or neighboring patient anatomy can change in size or relative position due to tumor shrinkage, patient weight loss, and intra-fraction motion of the patient or internal anatomy of the patient. The X-ray imaging systems currently employed for IGRT are onboard imaging systems and fixed room-based systems. Onboard imaging systems are mounted on and rotate with the treatment gantry, while fixed room-based systems usually include a pair of room-mounted X-ray imagers.

Onboard imaging systems can be configured with a single X-ray imager, which includes one X-ray source and one X-ray detector, or a pair of stereo X-ray imagers, each with a dedicated X-ray source and X-ray detector. Onboard imaging systems that have a single X-ray source and detector have a limited ability to enable detection of internal anatomy motion in three dimensions for multiple reasons. First, the radiographs produced by such imaging systems are two-dimensional snapshot images of patient anatomy that provide no three-dimensional position information. Consequently, changes in position of an anatomical structure, tumor, or other region of interest along the viewing direction of the X-ray imaging system is difficult to detect. Second, to collect three-dimensional position information with such imaging systems, multiple radiographs must be acquired, and each such radiograph is necessarily taken from a different angle and at a different time as the gantry rotates through the treatment arc. As a result, feature detection and matching based on such radiographs is much more challenging, less reliable, and inherently includes significant latency. Third, many radiographs acquired by onboard imaging systems are acquired from a point of view in which critical anatomical features are partially or completely blocked by high-density anatomy (e.g., bony structures), or highly attenuating anatomical features (e.g., the shoulder). These radiographs provide limited three-dimensional information and therefore have limited utility for motion detection. Stereo onboard imaging systems can more quickly provide three-dimensional position information, but in many instances can still be blocked by certain patient anatomy. Further, stereo onboard imaging systems add considerable complexity and cost to a radiation therapy system by consuming limited space on the gantry, adding weight to the load that must be supported and precisely rotated by the gantry, and increasing on-gantry power and cooling requirements.

Fixed room-based X-ray imaging systems typically include stereo X-ray imaging systems, and therefore are designed to detect patient motion detection based on three-dimensional position information derived from stereo X-ray imaging. However, in many instances, fixed room-based X-ray imaging systems are subject to occlusion by gantry-mounted components, such as the linear accelerator and gantry-mounted X-ray imaging systems. Further, fixed room-based X-ray imaging systems are typically separate systems from the radiation therapy system. As a result, a complex process of integrating communications between the radiation therapy system and a fixed room-based X-ray imaging system is required. Alternatively, room-based X-ray imaging systems can be mounted on one or more robotic arms to avoid occlusion by gantry-mounted components. However, for such systems to be feasible, a robot arm capable of highly precise movement is required, adding significant complexity and expense to a radiation therapy system. In addition, a complex control system for the robot arm and integrated communications between the radiation therapy system and the robot arm are required.

Accordingly, there is a need in the art for improved systems and methods for detecting patient motion with a radiation therapy system.

### C-Arm Gantry Embodiments

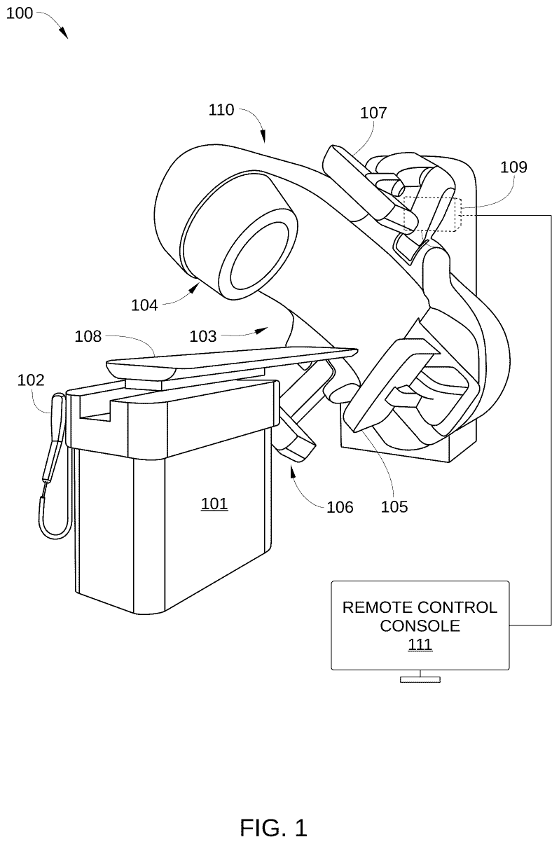

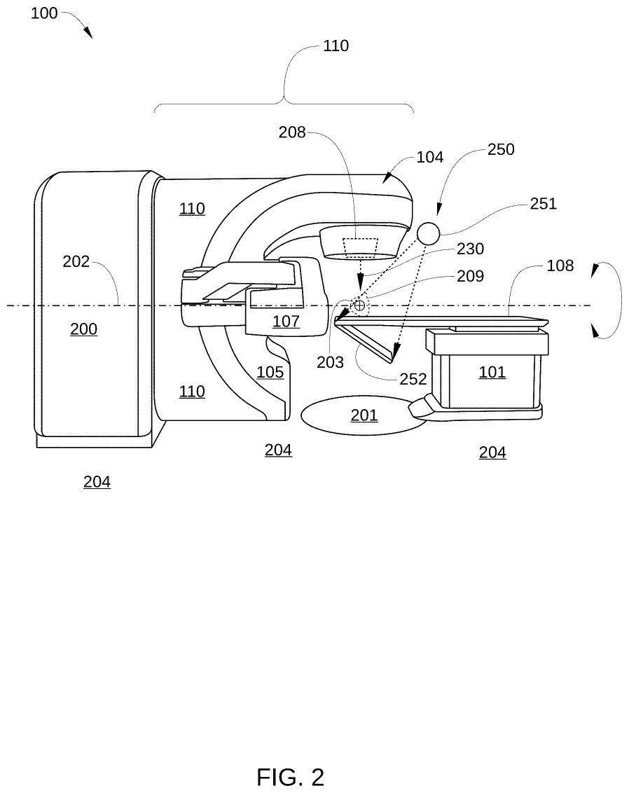

FIG. 1 is a perspective view of a radiation therapy system, according to various embodiments. Radiation therapy (RT) system 100 is a radiation system that may be configured to detect intra-fraction motion in near-real time using X-ray imaging techniques. Thus, in some embodiments, RT system 100 is configured to provide stereotactic radiosurgery and precision radiotherapy for lesions, tumors, and conditions anywhere in the body where radiation treatment is indicated. As such, RT system 100 can include one or more of a linear accelerator (LINAC) 104 that generates an MV treatment beam of high energy X-rays or other radiation, one or more kilovolt (kV) imaging X-ray sources 106, one or more imaging panels 107 (e.g., an X-ray imager), a mega-Volt (MV) electronic portal imaging device (EPID) 105 and a supplemental X-ray imaging system. The supplemental X-ray imaging system is described below in conjunction with FIG. 2. In the embodiment illustrated in FIG. 1, RT system 100 is configured with a C-arm gantry 110, which in some embodiments is a rotatable gantry capable of infinite rotation via a slip ring connection. In the embodiment illustrated in FIG. 1, imaging panel 107 is depicted as a planar device, whereas in other embodiments, imaging panel 107 can have a curved configuration.

In some embodiments, RT system 100 is capable of X-ray imaging of a target volume immediately prior to and/or during application of an MV treatment beam. Consequently, RT system 100 can perform an image-guided radiation therapy (IGRT) and/or an intensity-modulated radiation therapy (IMRT) process using the X-ray imaging generated by RT system 100. For example, in some embodiments, such processes can include kV imaging of the target volume in conjunction with the supplemental X-ray imaging system. Alternatively or additionally, in some embodiments, such processes can include imaging generated by the MV treatment beam in conjunction with the supplemental X-ray imaging system.

RT system 100 may include one or more touchscreens (not shown) for patient information verification, couch motion controls 102, a radiation area 103, a couch-positioning assembly 101, a treatment couch 108 disposed on couch-positioning assembly 101, and an image acquisition and treatment control computer 109, all of which are disposed within a treatment room. RT system 100 further includes a remote control console 111, which is disposed outside the treatment room and enables treatment delivery and patient monitoring from a remote location. Couch-positioning assembly 101 is configured to precisely position treatment couch 108 with respect to radiation area 103, including rotating treatment couch 108 about an isocenter of RT system 100. In some embodiments, a center of rotation (not shown) of couch-positioning assembly 101 is vertically aligned with the treatment isocenter. Motion controls 102 include input devices, such as buttons and/or switches, that enable a user to operate couch-positioning assembly 101 to automatically and precisely position treatment couch 108 to a predetermined location with respect to radiation area 103. Motion controls 102 also enable a user to manually position treatment couch 108 to a particular location, such as a planned treatment position for a patient or an anatomical target.

According to various embodiments, RT system 100 includes at least one onboard X-ray imaging system that is mounted on C-arm gantry 110 and a supplemental X-ray imaging system that is fixed in position while C-arm gantry 110 rotates about a treatment isocenter of RT system 100. In the embodiment illustrated in FIG. 1, one onboard X-ray imaging system that is mounted on C-arm gantry 110 of RT system 100 is implemented as imaging X-ray source 106 and imaging panel 107. In some embodiments, another onboard X-ray imaging system that is mounted on C-arm gantry 110 of RT system 100 is implemented as LINAC 104 (used as an X-ray source) and EPID 105 (used as an imaging panel). Further, RT system 100 includes a supplemental X-ray imaging system, which is described below in conjunction with FIG. 2.

FIG. 2 schematically illustrates a side view of RT system 100, according to various embodiments. As shown, RT system 100 includes a base stand 200, C-arm gantry 110, couch-positioning assembly 101 with treatment couch 108, and a supplemental X-ray imaging system 250. For clarity, in FIG. 2, EPID 105, imaging X-ray source 106, and imaging panel 107 are stowed and not deployed for use and imaging X-ray source 106 is not visible.

Base stand 200 is a fixed support structure for components of RT treatment system 100, including C-arm gantry 110 and a drive system (not shown) for rotatably moving C-arm gantry 110 about a horizontal rotation axis 202 and a treatment isocenter 203. Base stand 200 rests on and/or is fixed to a support surface that is external to RT treatment system 100, such as a floor 204 of an RT treatment facility. C-arm gantry 110 is rotationally coupled to base stand 200, for example via a bearing, and is a support structure on which various components of RT system 100 are mounted, including LINAC 104, EPID 105, imaging X-ray source 106, and imaging panel 107.

Couch-positioning assembly 101 is coupled to a turntable 201 that is mounted in floor 204 and rotates couch-positioning assembly 101 about treatment isocenter 203. Thus, couch-positioning assembly 101 in conjunction with turntable 201 enables rotational and linear motion of treatment couch 108 relative to isocenter 203. In some embodiments, couch-positioning assembly 101 is configured to rotate, pitch, roll, and/or translate treatment couch 108 relative to isocenter 203 to one or more treatment positions.

LINAC 104 is a radiation source, and typically includes one or more of an electron gun for generating electrons, an accelerating waveguide, an electron beam target, an electron beam transport means (such as a bending magnet) for directing the electron beam to the electron beam target, and/or a collimator assembly 208 for collimating and shaping a treatment beam 230 that originates from the electron beam target. Collimator assembly 208 typically includes one or more of a primary collimator that defines the largest available circular radiation field for treatment beam 230, a secondary collimator for providing a rectangular or square radiation field at isocenter 203 (for example via X-jaws and Y-jaws), and/or a multileaf collimator (MLC) for conforming treatment beam 230 to a planning target volume (PTV) or other anatomical target. In other embodiments, LINAC 104 can be any other radiation source suitable for radiation therapy.

During operation of RT treatment system 100, C-arm gantry 110 rotates about radiation area 103 (shown in FIG. 1) and isocenter 203 when actuated by the drive system for rotatably moving C-arm gantry 110 about horizontal rotation axis 202. Imaging X-ray source 106 (shown in FIG. 1) is configured to direct a conical beam of X-rays, referred to herein as imaging X-rays (not shown in FIG. 2 for clarity), through isocenter 203 of RT system 100 to imaging panel 107. In some instances, isocenter 203 corresponds to the location of a target volume 209 to be treated, such as a PTV, a gross tumor volume (GTV), a clinical target volume (CTV), and/or an internal target volume (ITV), among others. In other instances, target volume 209 (or a group of target volumes 209) is located off-isocenter. For example, in such instances, a group of multiple target volumes 209 may be treated in a single patient. In such instances, isocenter 203 can correspond to the location of a center of mass of the group of multiple target volumes 209, or some other suitable location proximate to the group of multiple target volumes 209.

During radiation treatment, LINAC 104 is configured to generate treatment beam 230, which can include high-energy radiation (for example MV X-rays or MV electrons). In other embodiments, treatment beam 230 includes electrons, protons, and/or other heavy charged particles, ultra-high dose rate X-rays (e.g., for FLASH radiotherapy), and/or microbeams for microbeam radiation therapy. In some embodiments, as treatment beam 230 is directed to isocenter 203 while C-arm gantry 110 rotates through a treatment arc, image acquisitions can be performed via EPID 105 to generate image data for target volume 209. For example, in such embodiments, EPID 105 generates one or more projection images of target volume 209 and/or a region of patient anatomy surrounding target volume 209.

According to various embodiments, supplemental X-ray imaging system 250 includes an X-ray imaging source 251 and an imaging panel 252 for acquiring X-ray images of and/or generating image information associated with isocenter 203 and/or target volume 209. X-ray imaging source 251 can be any technically feasible X-ray source, such as a kV imaging source. Imaging panel 252 can be any technically feasible X-ray imaging panel or flat-panel detector (PFD), including a direct-imaging panel or an indirect imaging panel.

In the embodiments, supplemental X-ray imaging system 250 is generally fixed in position while C-arm gantry 110 rotates about treatment isocenter 203. Thus, X-ray imaging source 251 and an imaging panel 252 are not mounted on C-arm gantry 110. Instead, X-ray imaging source 251 and/or imaging panel 252 is mounted on treatment couch 108, couch-positioning assembly 101, or a surface external to C-arm gantry or treatment couch 108, such as a location on floor 204 of a treatment room or treatment facility containing RT system 100 or a location on a ceiling of the treatment room or facility containing RT system 100. In the embodiment illustrated in FIG. 2, imaging panel 252 is coupled to treatment couch 108 and X-ray imaging source 251 is coupled to either treatment couch 108, couch-positioning assembly 101, or a ceiling of the treatment room or facility containing RT system 100.

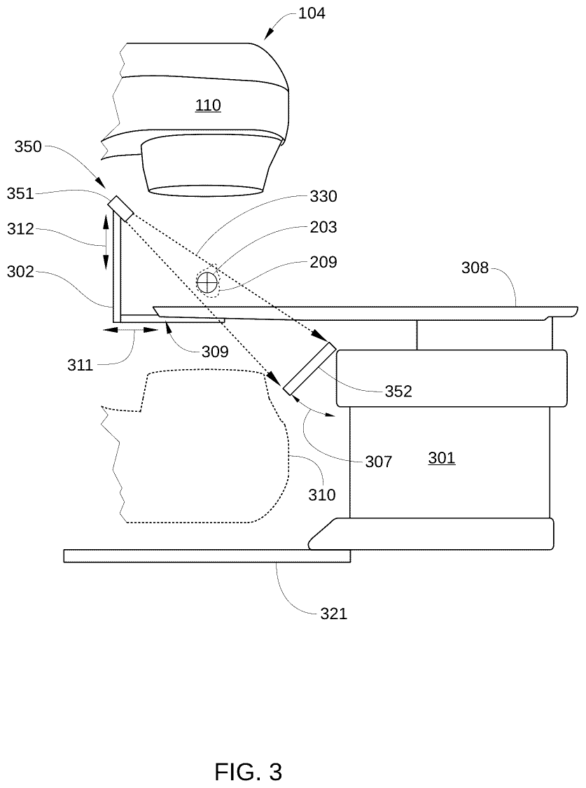

In the embodiment illustrated in FIG. 2, X-ray imaging source 251 is positioned above isocenter 203 and imaging panel 252 is positioned below isocenter 203. In such embodiments, imaging panel 252 can be mounted on couch-positioning assembly 101, treatment couch 108, a surface of turntable 201, or a surface of floor 204. Examples of such embodiments are described below in conjunction with FIGS. 3-6.

FIG. 3 is a conceptual side view of a couch-positioning assembly 301 and a supplemental X-ray imaging system 350, in which an imaging panel 352 of supplemental X-ray imaging system 350 is mounted on couch-positioning assembly 301, according to various embodiments. For reference, a portion of C-arm gantry 110 that includes LINAC 104 is included in FIG. 3. Also shown is a region 310 (dashed lines) that is swept by C-arm gantry 110 when C-arm gantry rotates about isocenter 203. In some embodiments, couch-positioning assembly 301 can be consistent with couch-positioning assembly 101 of FIGS. 1 and 2, and X-ray imaging system 350 can be consistent with X-ray imaging system 250 of FIG. 2.

Supplemental X-ray imaging system 350 includes an X-ray source 351 and imaging panel 352. As shown, X-ray source 351 is mounted above treatment couch 308, and imaging panel 352 is mounted on couch-positioning assembly 301. In some embodiments, X-ray source 351 and imaging panel 352 are each fixed in position relative to treatment couch 308. For example, X-ray source 351 can be coupled to treatment couch 308 via a support arm 302 that is coupled to treatment couch 308, and imaging panel 352 can be mounted onto a surface of either treatment couch 308 or couch-positioning assembly 301. Thus, in such embodiments, as a turntable 321 rotates couch-positioning assembly 301 and treatment couch 308 about isocenter 203, X-ray source 351 rotates with treatment couch 308. As a result, supplemental X-ray imaging system 350 has a fixed view of isocenter 203 and/or target volume 209 that is independent of the angle at which turntable 321 has rotated treatment couch 308 and couch-positioning assembly 301 about isocenter 203. Consequently, during an IGRT process, supplemental X-ray imaging system 350 can acquire an unobstructed X-ray image of isocenter 203 and/or target volume 209 in addition to an X-ray image acquired by an onboard imaging system, such as imaging X-ray source 106 and imaging panel 107.

The operation of supplemental X-ray imaging system 350 is controlled by a controller of RT system 100 (e.g., image acquisition and treatment control computer 109 in FIG. 1). As a result, supplemental X-ray imaging system 350 can generate image information for isocenter 203 and/or target volume 209 (e.g., acquire an X-ray image) synchronously with the onboard imaging system generating image information for isocenter 203 and/or target volume 209. In this disclosure, the term “synchronously” has at least the following meanings. In some embodiments, when supplemental X-ray imaging system 350 generates image information synchronously with the onboard imaging system, supplemental X-ray imaging system 350 generates image information for isocenter 203 and/or target volume 209 simultaneously with the onboard imaging system generating image information. In other embodiments, when supplemental X-ray imaging system 350 generates image information synchronously with the onboard imaging system, supplemental X-ray imaging system 350 and the onboard imaging system generate image information for isocenter 203 and/or target volume 209 sequentially but within a certain time interval. Thus, in such embodiments, supplemental X-ray imaging system 350 generates image information for isocenter 203 and/or target volume 209 at a first time and the onboard imaging system generates image information for isocenter 203 and/or target volume 209 at a second time that is offset from the first time by this certain time interval. In some embodiments, the first time is offset from the second time by a time interval in which C-arm gantry 110 cannot rotate through a sufficient arc to significantly or noticeably affect imaging. For example, the first time can be on the order of a few milliseconds up to about 10 milliseconds before or after the second time. Alternatively or additionally, in some embodiments, the first time is offset from the second time by a time interval in which anatomical motion (e.g., cardiac and/or respiration motion) cannot significantly or noticeably affect imaging. For example, the first time can be on the order of a few milliseconds before or after the second time. In embodiments in which supplemental X-ray imaging system 350 and the onboard imaging system generate image information for isocenter 203 and/or target volume 209 sequentially but within a certain time interval, isocenter 203 and/or target volume 209 is effectively imaged simultaneously by supplemental X-ray imaging system 350 and the onboard imaging system, but supplemental X-ray imaging system 350 is not affected by X-ray scatter from the onboard imaging system and vice versa. In addition, in such embodiments, the power consumption associated with generating image information with supplemental X-ray imaging system 350 and the power consumption associated with generating image information with the onboard imaging system are offset in time, thereby reducing peak power consumption of RT system 100. Furthermore, in such embodiments, due to the time interval separating imaging by supplemental X-ray imaging system 350 and imaging by the onboard imaging system, anatomical motion does not have a significant impact on image quality.

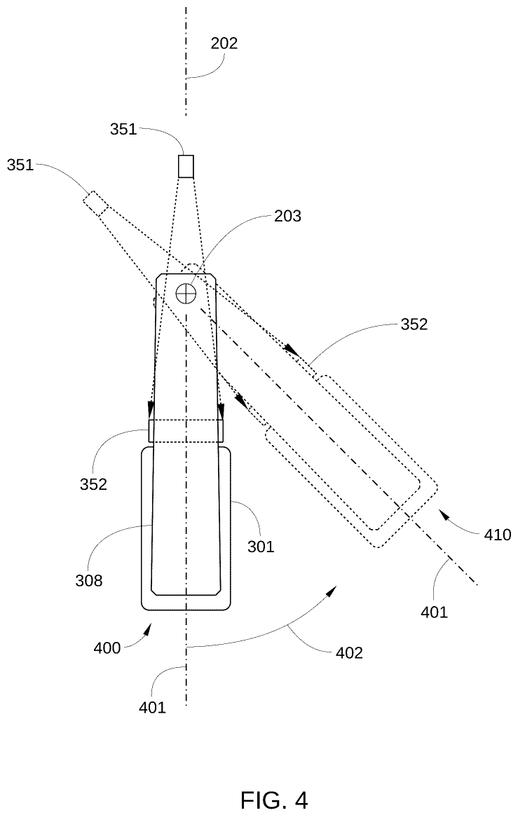

In the embodiment illustrated in FIG. 3, X-ray source 351 and imaging panel 352 are positioned so that a line of sight 330 between X-ray source 351 and imaging panel 352 is disposed outside of region 310, which is swept by C-arm gantry 110 and components mounted thereon when C-arm gantry rotates about treatment isocenter 203. As a result, supplemental X-ray imaging system 350 is not obstructed by C-arm gantry 110, LINAC 104, or any other components mounted on C-arm gantry when C-arm gantry 110 rotates about treatment isocenter 203 during treatment. In some embodiments, to facilitate line of sight 330 between X-ray source 351 and imaging panel 352, X-ray source 351 and imaging panel 352 are positioned to be aligned with treatment couch 308. One such embodiment is described below in conjunction with FIG. 4.

FIG. 4 schematically illustrates a plan view of treatment couch 308, couch-positioning assembly 301, and supplemental X-ray imaging system 350, according to various embodiments. For reference, isocenter 203 and horizontal rotation axis 202 are also shown in FIG. 4. In the embodiment illustrated in FIG. 4, X-ray source 351 and imaging panel 352 are positioned to be aligned with treatment couch 308. For example, in some embodiments, X-ray source 351 and imaging panel 352 are both positioned in a plane that is perpendicular to a support surface (e.g., a top surface) of couch 308 and parallel to a longitudinal axis 401 of treatment couch 308. Because X-ray source 351 and imaging panel 352 are coupled to treatment couch 308 and/or couch-positioning assembly 301, when couch-positioning assembly 301 is rotated to various treatment positions, X-ray source 351 and imaging panel 352 remain in alignment with treatment couch 308. Thus, X-ray source 351 and imaging panel 352 are aligned with longitudinal axis 401 of treatment couch 308 independent of how treatment couch 308 and couch-positioning assembly 301 are rotated about isocenter 203. For example, X-ray source 351 and imaging panel 352 are aligned with longitudinal axis 401 when treatment couch 308 and couch-positioning assembly 301 are in a neutral position 300, in which treatment couch 308 is in line with horizontal rotation axis 202 of C-arm gantry 110. Similarly, X-ray source 351 and imaging panel 352 are also aligned with longitudinal axis 401 when treatment couch 308 and couch-positioning assembly 301 are in a rotated position 410 (dashed lines), in which treatment couch 308 is rotated 45 degrees in a rotation direction 402 from neutral position 400.

Returning to FIG. 3, in some embodiments, support arm 302 can be implemented as an adjustable arm or a replaceable arm that enables repositioning of X-ray source 351, for example in a horizontal direction 311 and/or a vertical direction 312. In such embodiments, support arm 302 can reposition X-ray source 351 relative to treatment couch 308 and/or imaging panel 352 for different imaging applications. For example, to facilitate imaging of a head of a patient, an end 309 of treatment couch 308 may be positioned proximate to isocenter 203, and to facilitate imaging of a chest region of a patient, end 309 of treatment couch 308 may be extended past isocenter 203 in horizontal direction 311. In each case, when support arm 302 is implemented as an adjustable arm or a replaceable arm, the position and/or orientation of X-ray source 351 can be adjusted to facilitate imaging during an IGRT process to provide an appropriate viewing angle for the region of interest of patient anatomy. Additionally or alternatively, in some embodiments, imaging panel 352 can be deployed in a plurality of angles 307 relative to couch-positioning assembly 301, as shown. In such embodiments, imaging panel 352 can be angled appropriately to facilitate imaging when X-ray source 351 is positioned in different vertical or horizontal locations relative to isocenter 203 via adjustable or replaceable support arm 302.

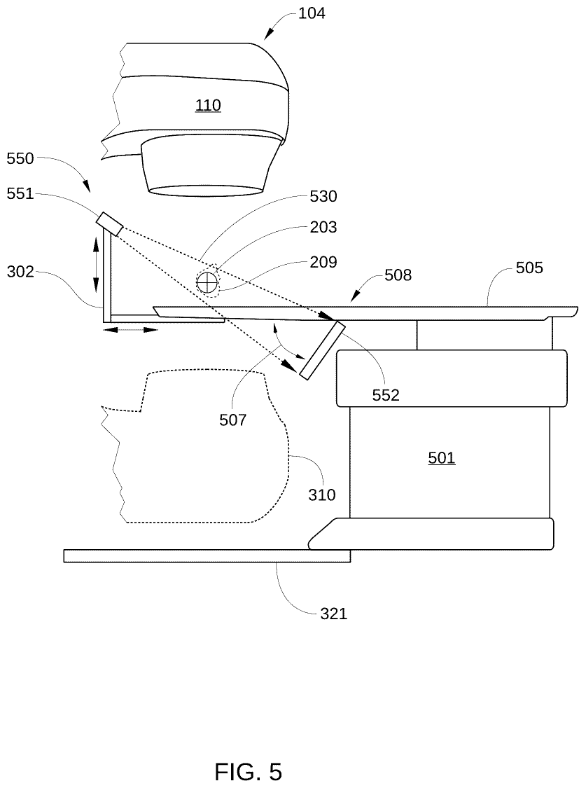

In some embodiments, when an X-ray source of a supplemental X-ray imaging system is positioned above isocenter 203 and an imaging panel of the supplemental X-ray imaging system is positioned below isocenter 203, the imaging panel can be mounted on a treatment couch of the radiation therapy system. One such embodiment is described below in conjunction with FIG. 5.

FIG. 5 is a conceptual side view of a couch-positioning assembly 501 and a supplemental X-ray imaging system 550, in which an imaging panel 552 of supplemental X-ray imaging system 550 is mounted on a treatment couch 508, according to various embodiments. For reference, a portion of C-arm gantry 110 that includes LINAC 104 is included in FIG. 5. Also shown is region 310 (dashed lines), which is swept by C-arm gantry 110 when C-arm gantry rotates about isocenter 203. In some embodiments, couch-positioning assembly 501 can be consistent with couch-positioning assembly 101 of FIGS. 1 and 2, and X-ray imaging system 550 can be consistent with X-ray imaging system 250 of FIG. 2.

Supplemental X-ray imaging system 550 includes an X-ray source 551 and imaging panel 552. As shown, X-ray source 551 is disposed above a support surface 505 of treatment couch 508 and mounted on treatment couch 508, while imaging panel 552 is disposed below support surface 505 and mounted on treatment couch 508. In some embodiments, X-ray source 551 and imaging panel 552 are each fixed in position relative to treatment couch 508. For example, in some embodiments, X-ray source 551 can be coupled to treatment couch 508 via support arm 302, and imaging panel 552 can be mounted onto a surface of treatment couch 308. Thus, in such embodiments, supplemental X-ray imaging system 550 has a fixed view of isocenter 203 and/or target volume 209 that is independent of the angle at which turntable 321 has rotated treatment couch 508 and couch-positioning assembly 501 about isocenter 203. Furthermore, similar to supplemental X-ray imaging system 350 in FIG. 3, the operation of supplemental X-ray imaging system 550 is controlled by a controller of RT system 100. As a result, supplemental X-ray imaging system 550 can generate image information for isocenter 203 and/or target volume 209 synchronously with the onboard imaging system generating image information for isocenter 203 and/or target volume 209. Also similar to supplemental X-ray imaging system 350, X-ray source 551 and imaging panel 552 are positioned so that a line of sight 530 between X-ray source 551 and imaging panel 552 is disposed outside of region 310.

In some embodiments, imaging panel 552 can be deployed in a plurality of angles 507 relative to couch-positioning assembly 501, as shown. In such embodiments, imaging panel 552 can be angled appropriately to facilitate imaging when X-ray source 551 is positioned in different vertical or horizontal locations relative to isocenter 203 via adjustable or replaceable support arm 302.

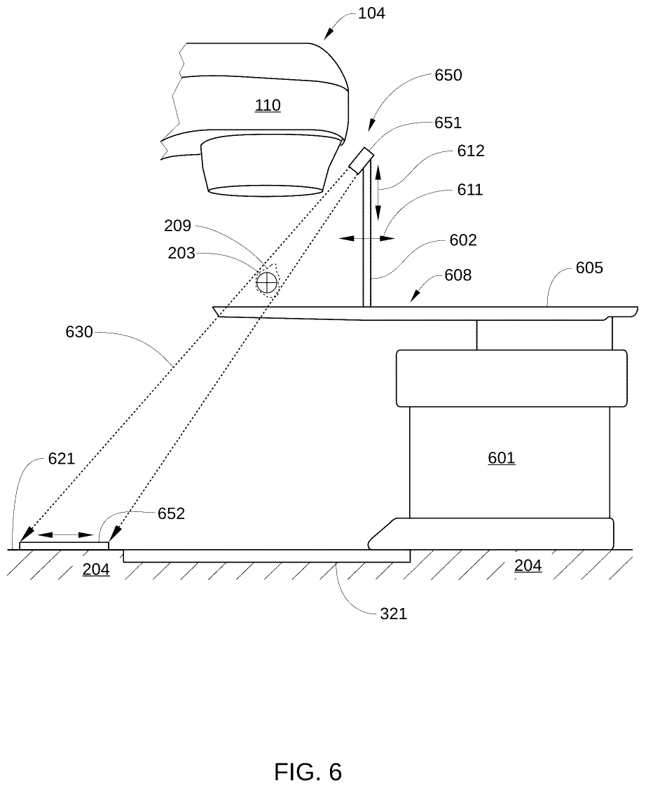

In some embodiments, when an X-ray source of a supplemental X-ray imaging system is positioned above isocenter 203 and an imaging panel of the supplemental X-ray imaging system is positioned below isocenter 203, the imaging panel can be mounted on a surface of a turntable of the radiation therapy system. One such embodiment is described below in conjunction with FIG. 6.

FIG. 6 is a conceptual side view of a couch-positioning assembly 601 and a supplemental X-ray imaging system 650, in which an imaging panel 652 of supplemental X-ray imaging system 650 is mounted on a surface 621 of floor 204 or a surface of turntable 321, according to various embodiments. For reference, a portion of C-arm gantry 110 that includes LINAC 104 is included in FIG. 6. In some embodiments, couch-positioning assembly 601 can be consistent with couch-positioning assembly 101 of FIGS. 1 and 2, and X-ray imaging system 650 can be consistent with X-ray imaging system 250 of FIG. 2.

Supplemental X-ray imaging system 650 includes an X-ray source 651 and imaging panel 652. As shown, X-ray source 651 is disposed above a support surface 605 of treatment couch 608 and mounted on treatment couch 608, while imaging panel 652 is disposed below support surface 605 and mounted on surface 621 of floor 204. Alternatively, in some embodiments, imaging panel 652 is mounted on a surface of turntable 321. In some embodiments, X-ray source 651 and imaging panel 652 are each fixed in position relative to treatment couch 608. For example, in some embodiments, X-ray source 651 can be coupled to treatment couch 608 via a support arm 602, and imaging panel 652 can be mounted on surface 621 of floor 204. Thus, in such embodiments, supplemental X-ray imaging system 650 provides an additional view of isocenter 203 and/or target volume 209. Similar to supplemental X-ray imaging system 350 in FIG. 3, the operation of supplemental X-ray imaging system 650 is controlled by a controller of RT system 100. As a result, supplemental X-ray imaging system 650 can generate image information for isocenter 203 and/or target volume 209 synchronously with the onboard imaging system generating image information for isocenter 203 and/or target volume 209. In an alternative embodiment, imaging panel 652 is disposed above support surface 605 of treatment couch 608 and mounted on treatment couch 608, while X-ray source 651 is disposed below support surface 605 and mounted on surface 621 of floor 204.

In some embodiments, support arm 602 can be consistent with support arm 302 of FIG. 3. In other embodiments, to cause a line of sight 630 between X-ray source 651 and imaging panel 652 to be unobstructed by LINAC 104 and/or other components mounted on C-arm gantry 110, support arm 602 can be a robotic arm that translates and/or rotates X-ray source 651 to different positions when C-arm gantry 110 has rotated to certain positions. In such embodiments, support arm 602 can include a suitable number of robotic joints (or “axes”) and links to enable the positioning of X-ray source 651 appropriately during operation. In such embodiments, support arm 602 can include one or more rotary joints, linear joints, twisting joints, revolute joints, spherical joints, and/or cylindrical joints. Alternatively or additionally, in some embodiments, imaging panel 652 can be movably mounted on surface 621, and therefore can be deployed in a plurality of locations relative to X-ray source 651. In such embodiments, imaging panel 652 can be repositioned to cause line of sight 630 between X-ray source 651 and imaging panel 652 to be unobstructed by LINAC 104 and/or other components mounted on C-arm gantry 110.

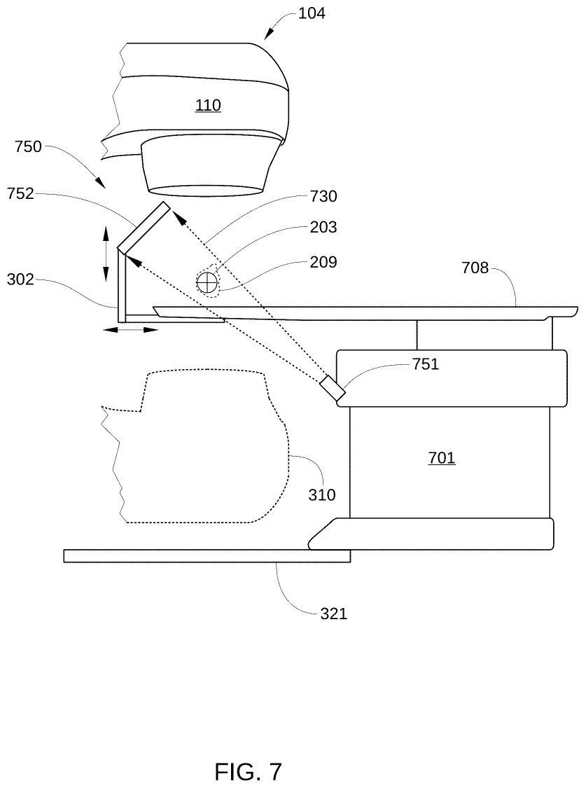

In some embodiments, an X-ray imaging source of a supplemental X-ray imaging system is positioned below isocenter 203 and an imaging panel of the supplemental X-ray imaging system is positioned above isocenter 203. In such embodiments, the X-ray imaging source can be mounted on a couch-positioning assembly or treatment couch of a radiation therapy system, a surface of a turntable of the radiation therapy system, or a surface of a floor of a treatment room or treatment facility containing the radiation therapy system. Examples of such embodiments are described below in conjunction with FIG. 7.

FIG. 7 is a conceptual side view of a couch-positioning assembly 701 and a supplemental X-ray imaging system 750, in which an X-ray source 751 of supplemental X-ray imaging system 750 is mounted on a treatment couch 708, according to various embodiments. For reference, a portion of C-arm gantry 110 that includes LINAC 104 is included in FIG. 7. Also shown is region 310 (dashed lines), which is swept by C-arm gantry 110 when C-arm gantry rotates about isocenter 203. In some embodiments, couch-positioning assembly 701 can be consistent with couch-positioning assembly 101 of FIGS. 1 and 2, and X-ray imaging system 750 can be consistent with X-ray imaging system 250 of FIG. 2.

Supplemental X-ray imaging system 750 includes X-ray source 751 and an imaging panel 752. As shown, imaging panel 752 is disposed above a support surface 705 of treatment couch 708 and mounted on treatment couch 708, while X-ray source 751 is disposed below support surface 705 and mounted on couch-positioning assembly 701. Alternatively, in some embodiments, X-ray source 751 is instead mounted on treatment couch 708. In some embodiments, X-ray source 751 and imaging panel 752 are each fixed in position relative to treatment couch 708. For example, in some embodiments, X-ray source 751 can mounted on couch-positioning assembly 701, and imaging panel 752 can be coupled to treatment couch 708 via support arm 302. Thus, in such embodiments, supplemental X-ray imaging system 750 has a fixed view of isocenter 203 and/or target volume 209 that is independent of the angle at which turntable 321 has rotated treatment couch 708 and couch-positioning assembly 701 about isocenter 203. Furthermore, similar to supplemental X-ray imaging system 350 in FIG. 3, the operation of supplemental X-ray imaging system 750 is controlled by a controller of RT system 100. As a result, supplemental X-ray imaging system 750 can generate image information for isocenter 203 and/or target volume 209 synchronously with the onboard imaging system generating image information for isocenter 203 and/or target volume 209. Also similar to supplemental X-ray imaging system 350, X-ray source 751 and imaging panel 752 are positioned so that a line of sight 730 between X-ray source 751 and imaging panel 752 is disposed outside of region 310. Further, because each of X-ray source 751 and imaging panel 752 is coupled to treatment couch 708 or couch-positioning assembly 701, when couch-positioning assembly 701 is rotated to various treatment positions, X-ray source 751 and imaging panel 752 remain in alignment with treatment couch 708.

### O-Ring Gantry Embodiments

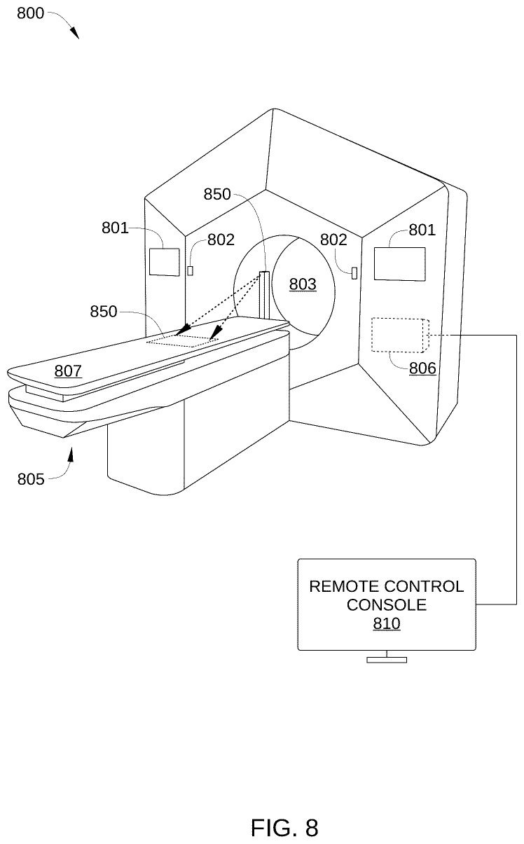

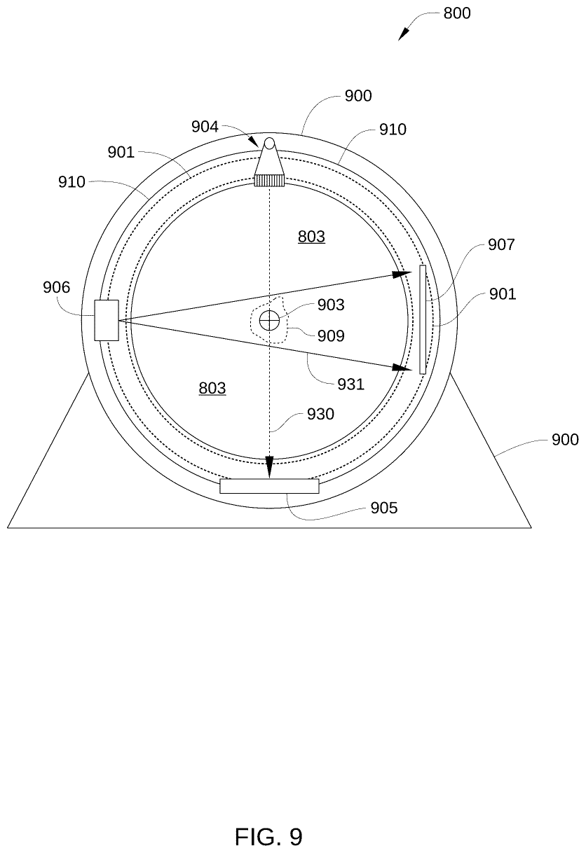

In the embodiment illustrated in FIGS. 1-7, RT system 100 includes C-arm gantry 110. In other embodiments, a radiation therapy system can be configured with a ring-based gantry that is disposed about an isocenter of the radiation therapy system. One such embodiment is described below in conjunction with FIGS. 8 and 9.

FIG. 8 is a perspective view of an RT system 800, according to various embodiments. In some embodiments, RT system 800 can be consistent with RT system 100 of FIGS. 1-7, except that RT system 800 is configured with a circular or ring-based gantry. As such, RT system 800 can include one or more touchscreens 801, couch motion controls (not shown), a bore 803, a base positioning assembly 805, a treatment couch 807 disposed on base positioning assembly 805, and an image acquisition and treatment control computer 806, all of which are disposed within a treatment room. RT system 800 further includes a remote control console 810, which is disposed outside the treatment room and enables treatment delivery and patient monitoring from a remote location. Base positioning assembly 805 is configured to precisely position treatment couch 807 with respect to bore 803, and the motion controls include input devices, such as button and/or switches, that enable a user to operate base positioning assembly 805 to automatically and precisely position treatment couch 807 to a predetermined location with respect to bore 803. The motion controls also enable a user to manually position treatment couch 807 to a predetermined location. According to various embodiments, RT system 800 further includes a supplemental X-ray imaging system 850, which can be consistent with various embodiments of supplemental X-ray imaging system 350 described above in conjunction with FIGS. 3-7.

FIG. 9 schematically illustrates a base stand 900 and gantry 910 of RT system 800, according to various embodiments. Covers, base positioning assembly 805, treatment couch 807, and other components of RT system 800 are omitted in FIG. 9 for clarity. Base stand 900 is a fixed support structure for components of RT system 800, including gantry 910 and a drive system 901 (dashed lines) for rotatably moving gantry 910. Base stand 900 rests on and/or is fixed to a support surface that is external to RT system 800, such as a floor of a radiotherapy treatment facility. Gantry 910 is rotationally coupled to base stand 900 and is a support structure on which various components of RT system 800 are mounted, including a LINAC 904, an EPID 905, an imaging X-ray source 906, and an X-ray imager 907.

During operation of RT system 800, drive system 901 rotationally actuates gantry 910, so that gantry 910 rotates about bore 803. LINAC 904 generates an MV treatment beam 930 of high energy X-rays (or in some embodiments electrons, protons, and/or other heavy charged particles, ultra-high dose rate X-rays (e.g., for FLASH radiotherapy) or microbeams for microbeam radiation therapy) and EPID 905 is configured to acquire X-ray images with treatment beam 930. Imaging X-ray source 906 is configured to direct a conical beam of X-rays, referred to herein as imaging X-rays 931, through an isocenter 903 of RT system 800 to X-ray imager 907, and isocenter 903 typically corresponds to the location of a target volume 909 to be treated. In the embodiment illustrated in FIG. 9, X-ray imager 907 is depicted as a planar device, whereas in other embodiments, X-ray imager 907 can have a curved configuration. According to various embodiments, supplemental X-ray imaging system 850 (not shown in FIG. 9) is also configured to acquire X-ray images of isocenter 903 and/or target volume 909.

RT system 800 includes a single X-ray imager and a single corresponding imaging X-ray source. In other embodiments, RT system 800 can include two or more X-ray imagers, each with a corresponding imaging X-ray source.

### Marker Tracking Using a Supplemental X-Ray Imaging System

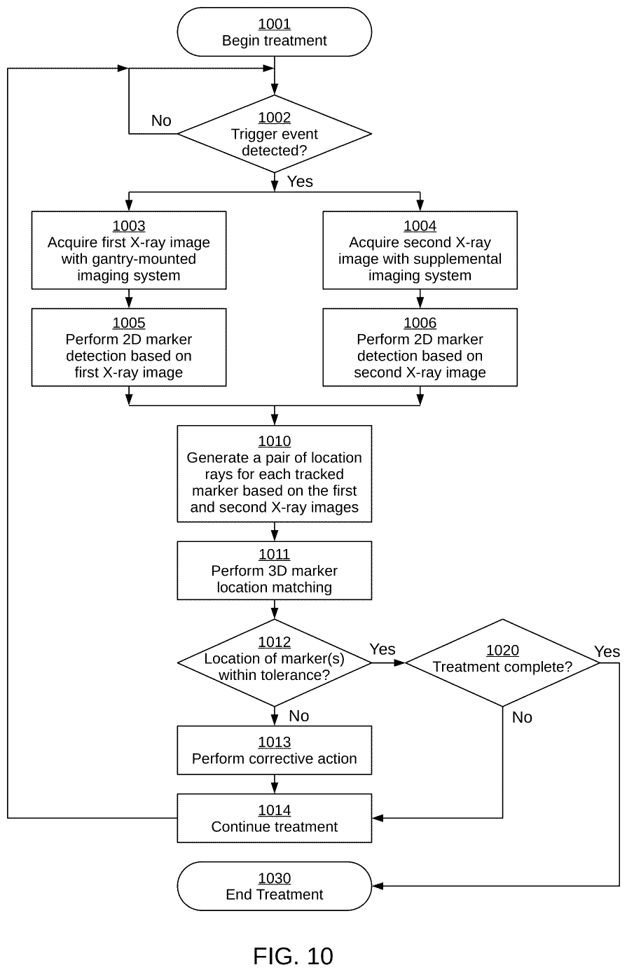

According to various embodiments, in a radiation therapy system, a supplemental X-ray imaging system is used in conjunction with an onboard X-ray imaging system to synchronously acquire a pair of two-dimensional (2D) X-ray images of a region of patient anatomy for marker tracking. For example, such embodiments can be employed during patient set up for an IGRT process and/or during an IGRT process. One such embodiment is described below in conjunction with FIG. 10.

FIG. 10 is a flowchart illustrating the steps of a computer-implemented process 1000 for marker tracking in a radiation therapy system, according to various embodiments. Computer-implemented process 1000 may include one or more operations, functions, or actions as illustrated by one or more of blocks 1001-1030. Although the blocks are illustrated in a specific order, these blocks may be performed in parallel, and/or in a different order than those described herein. Also, the various blocks may be combined into fewer blocks, divided into additional blocks, and/or eliminated based upon a specific implementation. Although the method is described in conjunction with radiation therapy systems 100 and 800, persons skilled in the art will understand that within the scope of the present disclosure any suitably configured radiation therapy system can perform computer-implemented process 1000.

In step 1001, an RT system (e.g., RT system 100 or 800) begins computer-implemented process 1000, which includes the treatment and imaging of a region of patient anatomy, such as a region that includes target volume 209. In some embodiments, computer-implemented process 1000 is performed as part of an IGRT, IMRT, adaptive radiotherapy (ART), or other imaging-related process. In some embodiments, computer-implemented process 1000 is performed as a treatment fraction. In step 1001, a controller of the RT system begins rotating C-arm gantry 110, for example at a specified rotational speed for delivery of treatment beam 230 to target volume 209. In step 1101, the controller of the RT system also directs treatment beam 230 to target volume 209 as appropriate. In some embodiments, treatment beam 230 is directed to target volume 209 continuously. In some embodiments, treatment beam 230 is directed to target volume 209 in pulses. In some embodiments, treatment beam 230 is directed to target volume 209 for a time interval in which rotatable gantry 210 has stopped rotating.

In step 1002, the controller determines whether a trigger event has been detected or occurred that indicates that imaging of the patient should take place. In some embodiments, the trigger event can be a time-based event, such as the expiration of a specified time interval. Alternatively or additionally, in some embodiments, the trigger event can be a gantry angle being reached by a rotatable gantry of the RT system or a specified dose level being reached. Alternatively or additionally, in some embodiments, the trigger event can be a signal generated by an optical system or other surrogate monitoring device indicating that intra-fraction motion of the patient and/or internal anatomy of the patient has occurred. Alternatively, in embodiments in which computer-implemented method 1000 is employed for patient setup prior to radiation therapy, the trigger event can be a manual input by a user or other indicator the patient setup process is to begin.

In step 1002, when the controller determines that a trigger event has been detected, computer-implemented process 1000 proceeds to step 1003; when the controller determines that no trigger event has been detected, computer-implemented process 1000 returns to step 1002 and treatment continues.

In step 1003, the controller causes a first X-ray image (or other X-ray image information) of a region of patient anatomy to be acquired with a gantry-mounted imaging system, such as imaging X-ray source 106 and imaging panel 107. In parallel with step 1003, in step 1004, the controller causes a second X-ray image (or other X-ray image information) of the region of patient anatomy to be acquired with a supplemental X-ray imaging system, such as supplemental X-ray imaging system 350. In some embodiments, the controller causes the first X-ray image and the second X-ray image to be acquired synchronously.

In step 1005, the controller performs 2D marker detection based on the first X-ray image. In some embodiments, the tracked marker(s) can include fiducial markers (e.g., implanted metallic objects) and/or specific features or landmarks of patient internal anatomy (e.g., boney structures), and are detected within the first X-ray image acquired by the onboard X-ray imaging system. In some embodiments, one or more tracked markers can correspond to and/or be disposed proximate target volume 209, treatment isocenter 203, an organ at risk (OAR), a breath-hold indicator (such as a position of the patient diaphragm), and/or any other region of interest (ROI) within the internal anatomy of the patient.

In some embodiments, in step 1005, the controller performs 2D marker detection by first generating a first angle-dependent digitally reconstructed radiograph (DRR) corresponding to the viewing angle of the gantry-mounted imaging system when acquiring the first X-ray image. For example, the first DRR can be a 2D view of certain tracked markers (fiducial markers and/or features of patient internal anatomy) that is generated based on a planning CT of a region of patient anatomy included in the first X-ray image and on imaging chain information associated with the gantry-mounted imaging system, such as gantry angle, imaging source power, detector geometry and orientation, and the like. The controller then identifies the tracked markers in the first X-ray image based on the locations of these tracked markers in the first DRR. In parallel with step 1005, in step 1006, the controller performs 2D marker detection based on the second X-ray image acquired by the supplemental X-ray imaging system. In some embodiments, the tracked markers are detected within the second X-ray image. In such embodiments, the controller generates a second angle-dependent DRR corresponding to the viewing angle of the supplemental X-ray imaging system when acquiring the second X-ray image. For example, the second DRR can be a 2D view of the tracked markers that is generated based on a planning CT of a region patient anatomy included in the second X-ray image and on imaging chain information associated with the supplemental imaging system, such as imaging source power and location, detector geometry and orientation, couch angle and position, and the like. The controller then identifies certain tracked markers in the second X-ray image based on the locations of these tracked markers in the second DRR.

In step 1010, the controller generates a pair of location rays for each tracked marker. For example, in some embodiments, for a particular tracked marker, a first location ray is generated in 3D space based on the first X-ray image and a second location ray is generated in 3D space based on the second X-ray image. Specifically, the position of the first location ray in 3D space is based on the location of the tracked marker in the first X-ray image and the viewing angle of the gantry-mounted imaging system when acquiring the first X-ray image, while the position of the second location ray in 3D space is based on the location of the tracked marker in the second X-ray image and the viewing angle of the supplemental imaging system when acquiring the second X-ray image. In such embodiments, the first location ray indicates the possible locations in 3D space that can possibly be occupied by the tracked marker from the point of view of the gantry-mounted imaging system, and the second location ray indicates the possible locations in 3D space that can possibly be occupied by the tracked marker from the point of view of the supplemental imaging system. This process is repeated for each tracked marker being tracked in computer-implemented method 1000.

In step 1011, the controller performs 3D marker location matching. For example, in some embodiments, for a particular tracked marker, the controller determines an intersection point in 3D space of the first location ray and the second location ray associated with that particular tracked marker. The location of the intersection point is then compared to the location of the tracked marker in a reference volume, such as a planning CT of the region of patient anatomy, a day-of-treatment 3D image (e.g., a setup cone-beam computed tomograph or “setup CBCT”) of the region of patient anatomy, a day-of-treatment 3D image of the region that has been deformably registered to a planning CT, or any other digital volume of the region of patient anatomy that can serve as a reference that indicates a target location for the tracked marker. The controller then determines an offset between the current (imaged) location of a particular tracked marker and the target location of the tracked marker, for example by comparing the intersection point in 3D space for that particular tracked marker with the target location of that tracked marker.

In step 1012, the controller determines whether the offset between the current location of each tracked marker and the corresponding target or expected location of each tracked marker is within a respective offset tolerance for the tracked marker. In some embodiments, there can be a different offset tolerance for each tracked marker. For example, the offset tolerance for tracked markers that correspond to target volume 209 or isocenter 203 may be different than the offset tolerance for tracked markers that correspond to a particular OAR. Similarly, the offset tolerance for tracked markers that correspond one particular OAR may be different than the offset tolerance for tracked markers that correspond to different OAR. When the controller determines that one or more offset tolerances have been exceeded, computer-implemented process 1000 proceeds to step 1013; when the controller determines that no offset tolerance has been exceeded, computer-implemented process 1000 proceeds to step 1020.

Step 1013 is performed in response to an offset tolerance for at least one tracked marker being exceeded. In step 1013, the controller performs one or more corrective actions. In some embodiments, the corrective action can include halting treatment, for example by halting delivery of treatment beam 230. Alternatively or additionally, in some embodiments, the corrective action can include modifying the planned treatment fraction to compensate for the change in location of the feature (e.g., target volume 209, isocenter 203, an OAR) corresponding to the out-of-tolerance tracked marker. In such embodiments, the location and intensity of dose delivered can be modified based on the change in location of one or more tracked markers. Alternatively or additionally, in some embodiments, the corrective action can include repositioning the patient relative to the radiation therapy system to compensate for the out-of-tolerance position of one or more tracked markers. In such embodiments, the treatment fraction can be completed without unloading the patient from the RT system. Computer-implemented process 1000 then proceeds to step 1014.

In step 1014, the current treatment continues and computer-implemented process 1000 returns to step 1002. In embodiments in which the corrective action performed in step 1013 is to halt treatment, the treatment associated with computer-implemented process 1000, such as a specific treatment fraction, is halted and computer-implemented process 1000 ends.

In step 1020, the controller determines whether the treatment associated with computer-implemented process 1000 is completed. If yes, computer-implemented process 1000 proceeds to step 1030 and computer-implemented process 1000 ends; if no, computer-implemented process 1000 proceeds to step 1014 and computer-implemented process 1000 continues.

It is noted that implementation of computer-implemented process 1000 enables more accurate determination of the position of one or more tracked markers in real time by an RT system due to use of multiple synchronous 2D X-ray images of a region of patient anatomy. In particular, an X-ray image of the region that is acquired by a supplemental X-ray imaging system of the RT system, as described herein, can be an X-ray image that is not subject to being occluded by a rotatable gantry of the RT system or blocked by high-density anatomy or highly attenuating anatomical features.

### 2D-3D Matching Using a Supplemental X-Ray Imaging System

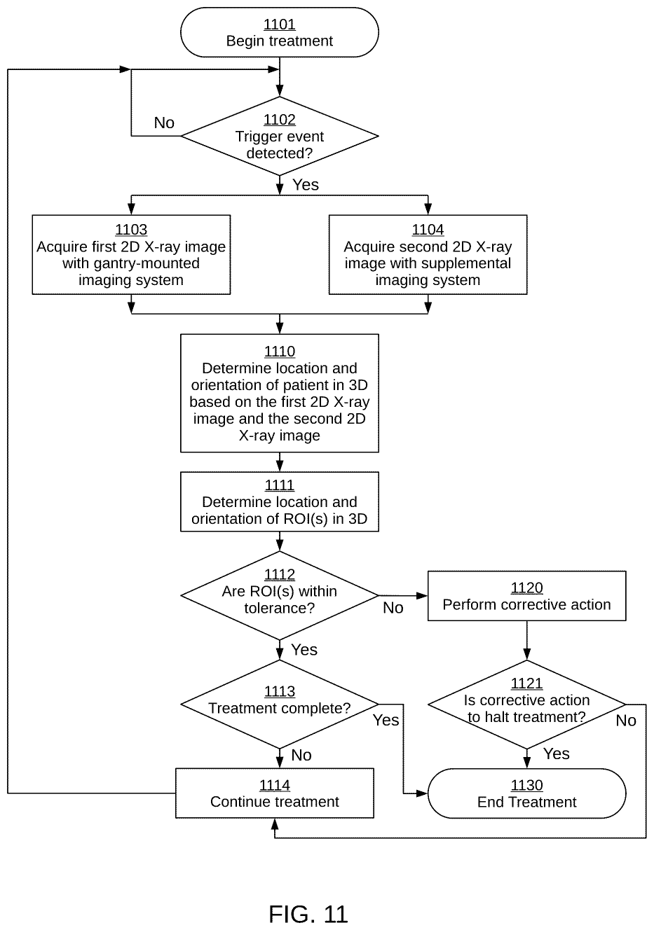

According to various embodiments, in a radiation therapy system, a supplemental X-ray imaging system is used in conjunction with an onboard X-ray imaging system to synchronously acquire a pair of two-dimensional (2D) X-ray images of a region of patient anatomy for 2D-3D matching. For example, such embodiments can be employed during patient set up for an IGRT process and/or during an IGRT process. One such embodiment is described below in conjunction with FIG. 11.

FIG. 11 is a flowchart illustrating the steps of a computer-implemented process 1100 for 2D-3D matching in a radiation therapy system, according to various embodiments. Computer-implemented process 1100 may include one or more operations, functions, or actions as illustrated by one or more of blocks 1101-1130. Although the blocks are illustrated in a specific order, these blocks may be performed in parallel, and/or in a different order than those described herein. Also, the various blocks may be combined into fewer blocks, divided into additional blocks, and/or eliminated based upon a specific implementation. Although the method is described in conjunction with radiation therapy systems 100 and 800, persons skilled in the art will understand that within the scope of the present disclosure any suitably configured radiation therapy system can perform computer-implemented process 1100.

In step 1101, an RT system (e.g., RT system 100 or 800) begins computer-implemented process 1100, which includes the treatment and imaging of a region of patient anatomy, such as a region that includes target volume 209. In some embodiments, computer-implemented process 1100 is performed as part of an IGRT, IMRT, adaptive radiotherapy (ART), or other imaging-related process. In some embodiments, computer-implemented process 1100 is performed as a treatment fraction. In step 1101, a controller of the RT system begins rotating C-arm gantry 110, for example at a specified rotational speed for delivery of treatment beam 230 to target volume 209. In step 1101, the controller of the RT system also directs treatment beam 230 to target volume 209 as appropriate. In some embodiments, treatment beam 230 is directed to target volume 209 continuously. In some embodiments, treatment beam 230 is directed to target volume 209 in pulses. In some embodiments, treatment beam 230 is directed to target volume 209 for a time interval in which rotatable gantry 210 has stopped rotating.

In step 1102, the controller determines whether a trigger event has been detected or occurred that indicates that imaging of the patient should take place. In some embodiments, the trigger event can be a time-based event, such as the expiration of a specified time interval. Alternatively or additionally, in some embodiments, the trigger event can be a gantry angle being reached by a rotatable gantry of the RT system or a specified dose level being reached. Alternatively or additionally, in some embodiments, the trigger event can be a signal generated by an optical system or other surrogate monitoring device indicating that intra-fraction motion of the patient and/or internal anatomy of the patient has occurred. Alternatively, in embodiments in which computer-implemented method 1100 is employed for patient setup prior to radiation therapy, the trigger event can be a manual input by a user or other indicator the patient setup process is to begin.

In step 1102, when the controller determines that a trigger event has been detected, computer-implemented process 1100 proceeds to step 1103; when the controller determines that no trigger event has been detected, computer-implemented process 1100 returns to step 1102 and treatment continues.

In step 1103, the controller causes a first X-ray image (or other X-ray image information) of a region of patient anatomy to be acquired with a gantry-mounted imaging system, such as imaging X-ray source 106 and imaging panel 107. In parallel with step 1103, in step 1104, the controller causes a second X-ray image (or other X-ray image information) of the region of patient anatomy to be acquired with a supplemental X-ray imaging system, such as supplemental X-ray imaging system 350. In some embodiments, the controller causes the first X-ray image and the second X-ray image to be acquired synchronously.

In step 1110, the controller performs 2D-3D matching. For example, in some embodiments, the controller determines the position in 3D space of the patient or a region of patient anatomy based on the first X-ray image of the region of patient anatomy acquired in step 1103 and the second X-ray image of the region of patient anatomy acquired in step 1104. In some embodiments, the controller applies a registration algorithm (e.g., rigid or non-rigid registration) to a reference volume of the region of patient anatomy based on the first X-ray image of the region of patient anatomy and the second X-ray image of the region of patient anatomy. For example, the reference volume can be a planning CT of the region of patient anatomy, a day-of-treatment 3D image of the region of patient anatomy (e.g., a setup CBCT), a day-of-treatment 3D image of the region that has been deformably registered to a planning CT, or any other digital volume of the region of patient anatomy that can serve as a reference volume. In such embodiments, by treating the patient or region of patient anatomy as a rigid body, the controller can determine the orientation and position of the patient or region of patient anatomy with respect to the RT system in terms of six degrees of freedom of the orientation. In step 1110, any suitable 2D-3D registration algorithm can be employed to determine the orientation and position of the patient or region of patient anatomy based on the first X-ray image of the region of patient anatomy and the second X-ray image of the region of patient anatomy. It is noted that, because the first X-ray image and the second X-ray image are synchronously acquired and are acquired with different viewing angles, the orientation and position of the patient or region of patient anatomy can be more accurately and quickly determined compared to prior art approaches. It is further noted that such benefits are present even when the viewing direction of the gantry-mounted X-ray imaging system is not orthogonal to the viewing direction of the supplemental X-ray imaging system.

In step 1111, the controller determines the location and/or orientation of one or more ROIs within the region of patient anatomy. In some embodiments, the ROIs can include one or more of target volume 209, isocenter 203, and/or various OARs. Generally, the controller determines the location and/or orientation of one or more ROIs based on the results of the 2D-3D registration performed in step 1110.

In step 1112, the controller determines whether the location and/or orientation of the one or more ROIs are within certain offset tolerances. For example, in some embodiments, for each ROI, the controller determines whether an offset between the current location and/or orientation of the ROI and the expected location and/or orientation of the ROI is within a respective offset tolerance for the ROI. In some embodiments, there can be a different offset tolerance for each tracked marker. For example, the offset tolerance for an ROI that corresponds to target volume 209 or isocenter 203 may be different than the offset tolerance for the ROI that correspond to a particular OAR. When the controller determines that the location and/or orientation of one or more ROIs exceeds the corresponding offset tolerance, computer-implemented process 1100 proceeds to step 1120; when the controller determines that the location and/or orientation of no ROIs exceeds the corresponding offset tolerance, computer-implemented process 1100 proceeds to step 1113.

In step 1113, the controller determines whether the treatment associated with computer-implemented process 1100 is completed. If yes, computer-implemented process 1100 proceeds to step 1130 and computer-implemented process 1100 ends; if no, computer-implemented process 1100 proceeds to step 1114. In step 1114, the current treatment continues and computer-implemented process 1100 returns to step 1102.

Step 1120 is performed in response to an offset tolerance for at least one ROI being exceeded. In step 1120, the controller performs one or more corrective actions. In some embodiments, the corrective action can include halting treatment, for example by halting delivery of treatment beam 230. Alternatively or additionally, in some embodiments, the corrective action can include modifying the planned treatment fraction to compensate for the change in location of the feature (e.g., target volume 209, isocenter 203, an OAR) corresponding to the out-of-tolerance ROI. In such embodiments, the location and intensity of dose delivered can be modified based on the change in location of one or more ROIs. Alternatively or additionally, in some embodiments, the corrective action can include repositioning the patient relative to the radiation therapy system to compensate for the out-of-tolerance position of one or more ROIs. In such embodiments, the treatment fraction can be completed without unloading the patient from the RT system. Computer-implemented process 1100 then proceeds to step 1121.

In step 1121, the controller determines whether the corrective action is to halt the current treatment associated with computer-implemented process 1100. If yes, computer-implemented process 1100 proceeds to step 1130 and computer-implemented process 1100 ends; if no, computer-implemented process 1100 proceeds to step 1114 and continues.

It is noted that implementation of computer-implemented process 1100 enables more accurate determination in real time by an RT system of the position and/or orientation of a patient. Due to the use of multiple synchronous 2D X-ray images of a region of patient anatomy, an RT system can more accurately determine the position and/or orientation of a patient and infer the position of one or more ROIs prior to and/or during treatment. In particular, an X-ray image of the region that is acquired by a supplemental X-ray imaging system of the RT system, as described herein, can be an X-ray image that is not subject to being occluded by a rotatable gantry of the RT system or blocked by high-density anatomy or highly attenuating anatomical features.

### Patient Model Matching Using a Supplemental X-Ray Imaging System

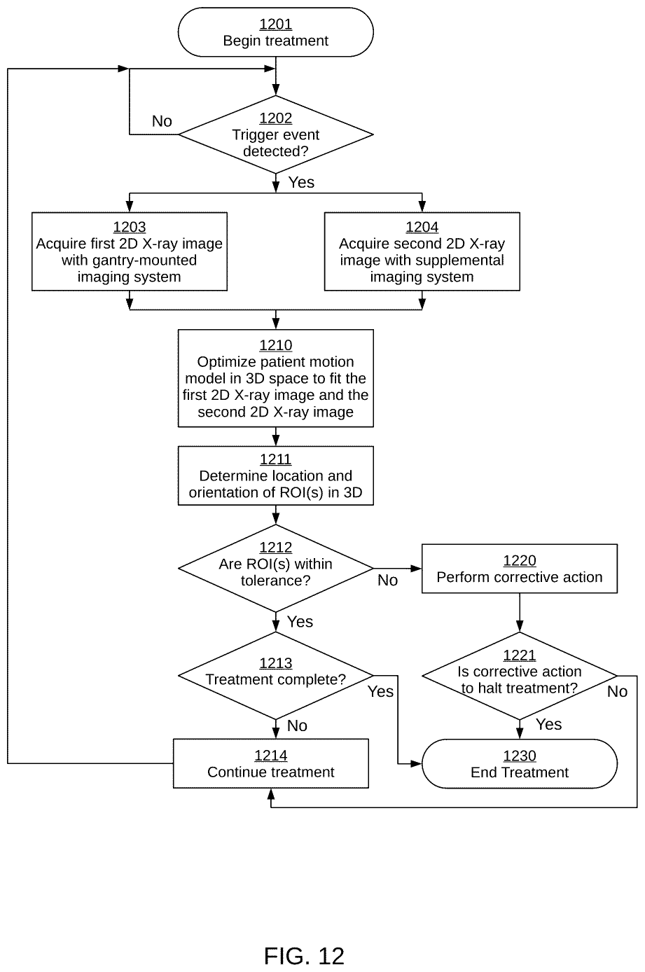

According to various embodiments, in a radiation therapy system, a supplemental X-ray imaging system is used in conjunction with an onboard X-ray imaging system to synchronously acquire pairs of 2D X-ray images of a region of patient anatomy that are then applied to a patient-motion model. For example, such embodiments can be employed during patient set up for an IGRT process and/or during an IGRT process. In the embodiments, image information for a patient is acquired by the onboard X-ray imaging system and the supplemental X-ray imaging system of a radiation therapy system and is applied to a motion model of patient deformation that has been developed for a patient. For example, a 4D motion model of patient cardiac and/or respiration motion can be developed. Through parameter fitting of the patient motion model to a synchronously acquired pair of 2D X-ray images of the patient, a real-time 3D representation of patient anatomy can be constructed that enables detection of motion of internal anatomy of the patient. One such embodiment is described below in conjunction with FIG. 12.

FIG. 12 is a flowchart illustrating the steps of a computer-implemented process 1200 for patient model matching in a radiation therapy system, according to various embodiments. Computer-implemented process 1200 may include one or more operations, functions, or actions as illustrated by one or more of blocks 1201-1230. Although the blocks are illustrated in a specific order, these blocks may be performed in parallel, and/or in a different order than those described herein. Also, the various blocks may be combined into fewer blocks, divided into additional blocks, and/or eliminated based upon a specific implementation. Although the method is described in conjunction with radiation therapy systems 100 and 800, persons skilled in the art will understand that within the scope of the present disclosure any suitably configured radiation therapy system can perform computer-implemented process 1200.

Prior to computer-implemented process 1200, a patient motion model is developed that can be employed in computer-implemented process 1200. In general, the patient motion model describes the overall deformation of the internal anatomy (or a specific region of internal anatomy) of a patient over time, for example during a respiration cycle. In some embodiments, the patient motion model is developed based on 4D (three spatial dimensions plus respiratory or cardiac phase) image information associated with a specific patient, such as 4D CT image information and/or 4D magnetic resonance imaging (MRI) image information. Alternatively or additionally, in some embodiments, the patient motion model is based on 4D image information associated with a specific population of patients. In either case, the patient motion model includes a plurality of parameters (e.g., lung volume) that together describe the patient motion state or breathing state in three spatial dimensions plus time.

In step 1201, an RT system (e.g., RT system 100 or 800) begins computer-implemented process 1200, which includes the treatment and imaging of a region of patient anatomy, such as a region that includes target volume 209. In some embodiments, computer-implemented process 1200 is performed as part of an IGRT, IMRT, adaptive radiotherapy (ART), or other imaging-related process. In some embodiments, computer-implemented process 1200 is performed as a treatment fraction. In step 1201, a controller of the RT system begins rotating C-arm gantry 110, for example at a specified rotational speed for delivery of treatment beam 230 to target volume 209. In step 1201, the controller of the RT system also directs treatment beam 230 to target volume 209 as appropriate. In some embodiments, treatment beam 230 is directed to target volume 209 continuously. In some embodiments, treatment beam 230 is directed to target volume 209 in pulses. In some embodiments, treatment beam 230 is directed to target volume 209 for a time interval in which rotatable gantry 210 has stopped rotating.

In step 1202, the controller determines whether a trigger event has been detected or occurred that indicates that imaging of the patient should take place. In some embodiments, the trigger event can be a time-based event, such as the expiration of a specified time interval. Alternatively or additionally, in some embodiments, the trigger event can be a gantry angle being reached by a rotatable gantry of the RT system or a specified dose level being reached. Alternatively or additionally, in some embodiments, the trigger event can be a signal generated by an optical system or other surrogate monitoring device indicating that intra-fraction motion of the patient and/or internal anatomy of the patient has occurred. Alternatively, in embodiments in which computer-implemented method 1200 is employed for patient setup prior to radiation therapy, the trigger event can be a manual input by a user or other indicator the patient setup process is to begin.

In step 1202, when the controller determines that a trigger event has been detected, computer-implemented process 1200 proceeds to step 1203; when the controller determines that no trigger event has been detected, computer-implemented process 1200 returns to step 1202 and treatment continues.

In step 1203, the controller causes a first X-ray image (or other X-ray image information) of a region of patient anatomy to be acquired with a gantry-mounted imaging system, such as imaging X-ray source 106 and imaging panel 107. In parallel with step 1203, in step 1204, the controller causes a second X-ray image (or other X-ray image information) of the region of patient anatomy to be acquired with a supplemental X-ray imaging system, such as supplemental X-ray imaging system 350. In some embodiments, the controller causes the first X-ray image and the second X-ray image to be acquired synchronously.

In step 1210, the controller optimizes the patient motion model in 3D space based on the first X-ray image of the region of patient anatomy acquired in step 1203 and the second X-ray image of the region of patient anatomy acquired in step 1204. In particular, the controller fits some or all model parameters to the first X-ray image and the second X-ray image, such as parameters representing an internal respiratory and/or cardiac motion state. In some embodiments, a registration algorithm can be employed to optimize the position and/or orientation of the model with respect to RT system 100.

For example, in some embodiments, the controller generates a first DRR of the region of patient anatomy using a 3D representation of the region derived from the patient motion model and imaging chain information for the gantry-mounted X-ray imaging system. The controller also generates a second DRR of the region of patient anatomy using the 3D representation of the region derived from the patient motion model and imaging chain information for the supplemental X-ray imaging system. The first DRR is compared to the first X-ray image and the second DRR is compared to the second X-ray image. Then, via an optimization process, one or more parameters of the patient motion model are iteratively modified until the latest iteration of the first DRR matches the first X-ray image and the latest iteration of the second DRR matches the second X-ray image.

In another example, in some embodiments, a specified feature within the region of patient anatomy (e.g., target volume 209) is determined based on the first X-ray image and the second X-ray image. For example, in some embodiments, the process described in steps 1110 and 1111 of computer-implemented process 1100 for determining the location of one or more ROIs can be employed to determine such a specified location. Alternatively, in some embodiments, the process described in steps 1003-1011 of computer-implemented process 1000 for determining the location of one or more tracked markers can be employed to determine such a specified location. Once the specified feature has been determined, the controller modifies one or more parameters of the patient motion model such that the position in 3D space of the specified feature within the model coincides with the currently determined position in 3D space of the specified feature.

In step 1211, based on the updated version of the patient motion model, the controller determines the location and/or orientation of one or more ROIs within the region of patient anatomy. In some embodiments, the ROIs can include one or more of target volume 209, isocenter 203, and/or various OARs.

In step 1212, the controller determines whether the location and/or orientation of the one or more ROIs are within certain offset tolerances. For example, in some embodiments, for each ROI, the controller determines whether an offset between the current location and/or orientation of the ROI and the expected location and/or orientation of the ROI is within a respective offset tolerance for the ROI. In some embodiments, there can be a different offset tolerance for each tracked marker. For example, the offset tolerance for an ROI that corresponds to target volume 209 or isocenter 203 may be different than the offset tolerance for the ROI that correspond to a particular OAR. When the controller determines that the location and/or orientation of one or more ROIs exceeds the corresponding offset tolerance, computer-implemented process 1200 proceeds to step 1220; when the controller determines that the location and/or orientation of no ROIs exceeds the corresponding offset tolerance, computer-implemented process 1200 proceeds to step 1213.

In step 1213, the controller determines whether the treatment associated with computer-implemented process 1200 is completed. If yes, computer-implemented process 1200 proceeds to step 1230 and computer-implemented process 1200 ends; if no, computer-implemented process 1200 proceeds to step 1214. In step 1214, the current treatment continues and computer-implemented process 1200 returns to step 1202.

Step 1220 is performed in response to an offset tolerance for at least one ROI being exceeded. In step 1220, the controller performs one or more corrective actions. In some embodiments, the corrective action can include halting treatment, for example by halting delivery of treatment beam 230. Alternatively or additionally, in some embodiments, the corrective action can include modifying the planned treatment fraction to compensate for the change in location of the feature (e.g., target volume 209, isocenter 203, an OAR) corresponding to the out-of-tolerance ROI. In such embodiments, the location and intensity of dose delivered can be modified based on the change in location of one or more ROIs. Alternatively or additionally, in some embodiments, the corrective action can include repositioning the patient relative to the radiation therapy system to compensate for the out-of-tolerance position of one or more ROIs. In such embodiments, the treatment fraction can be completed without unloading the patient from the RT system. Computer-implemented process 1200 then proceeds to step 1221.

In step 1221, the controller determines whether the corrective action is to halt the current treatment associated with computer-implemented process 1200. If yes, computer-implemented process 1200 proceeds to step 1230 and computer-implemented process 1200 ends; if no, computer-implemented process 1200 proceeds to step 1214 and continues.

It is noted that implementation of computer-implemented process 1200 enables more accurate determination in real time by an RT system of the overall deformation of the internal anatomy (or a specific region of internal anatomy) of a patient. Further, such deformation of the internal anatomy can be determined over time, for example during a respiration cycle. Due to the use of multiple synchronous 2D X-ray images of the region of patient anatomy, deformation of patient internal anatomy can be more accurately estimated based on a patient motion model.

In the embodiments described above in conjunction with FIGS. 10-12, synchronous use of an onboard imaging system and supplemental X-ray imaging system 350 is employed. In other embodiments, supplemental X-ray imaging system 350 can be employed without the onboard imaging system. For example, in some instances, dose to the patient can be spared by employing supplemental X-ray imaging system 350 in the herein-described embodiments without the onboard imaging system. Alternatively, or additionally, in some instances of the herein-described embodiments, deployment of the onboard imaging system can be problematic or impossible, for example due to clearance issues. In such instances, supplemental X-ray imaging system 350 is employed as described herein while the onboard imaging system is not used.

### Example Computing Device

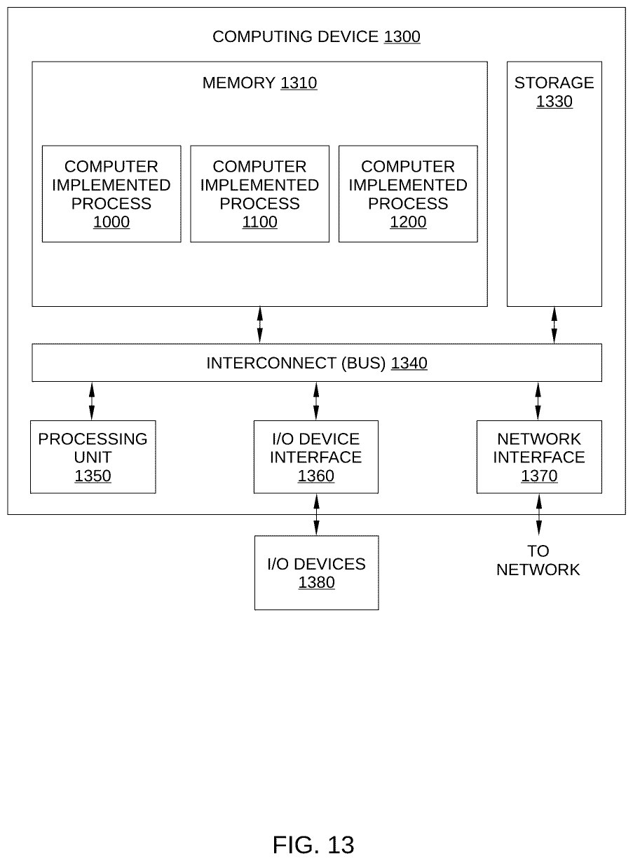

FIG. 13 is an illustration of a computing device 1300 configured to perform various embodiments of the present disclosure. For example, in some embodiments, computing device 1300 can be implemented as image acquisition and treatment control computer 109 of RT system 100 or image acquisition and treatment control computer 806 of RT system 800. Computing device 1300 may be a desktop computer, a laptop computer, a smart phone, or any other type of computing device suitable for practicing one or more embodiments of the present disclosure. In operation, computing device 1300 is configured to execute instructions associated with computer-implemented process 1000, computer-implemented process 1100, and/or computer-implemented process 1200, as described herein. It is noted that the computing device described herein is illustrative and that any other technically feasible configurations fall within the scope of the present disclosure.

As shown, computing device 1300 includes, without limitation, an interconnect (bus) 1340 that connects a processing unit 1350, an input/output (I/O) device interface 1360 coupled to input/output (I/O) devices 1380, memory 1310, a storage 1330, and a network interface 1370. Processing unit 1350 may be any suitable processor implemented as a central processing unit (CPU), a graphics processing unit (GPU), an application-specific integrated circuit (ASIC), a field programmable gate array (FPGA), any other type of processing unit, or a combination of different processing units, such as a CPU configured to operate in conjunction with a GPU or digital signal processor (DSP). In general, processing unit 1350 may be any technically feasible hardware unit capable of processing data and/or executing software applications, including computer-implemented process 1000, computer-implemented process 1100, and/or computer-implemented process 1200.

I/O devices 1380 may include devices capable of providing input, such as a keyboard, a mouse, a touch-sensitive screen, and so forth, as well as devices capable of providing output, such as a display device and the like. Additionally, I/O devices 1380 may include devices capable of both receiving input and providing output, such as a touchscreen, a universal serial bus (USB) port, and so forth. I/O devices 1380 may be configured to receive various types of input from an end-user of computing device 1300, and to also provide various types of output to the end-user of computing device 1300, such as displayed digital images or digital videos. In some embodiments, one or more of I/O devices 1380 are configured to couple computing device 1300 to a network.

Memory 1310 may include a random access memory (RAM) module, a flash memory unit, or any other type of memory unit or combination thereof. Processing unit 1350, I/O device interface 1360, and network interface 1370 are configured to read data from and write data to memory 1310. Memory 1310 includes various software programs that can be executed by processor 1350 and application data associated with said software programs, including computer-implemented process 1000, computer-implemented process 1100, and/or computer-implemented process 1200.

### Example Computer Program Product

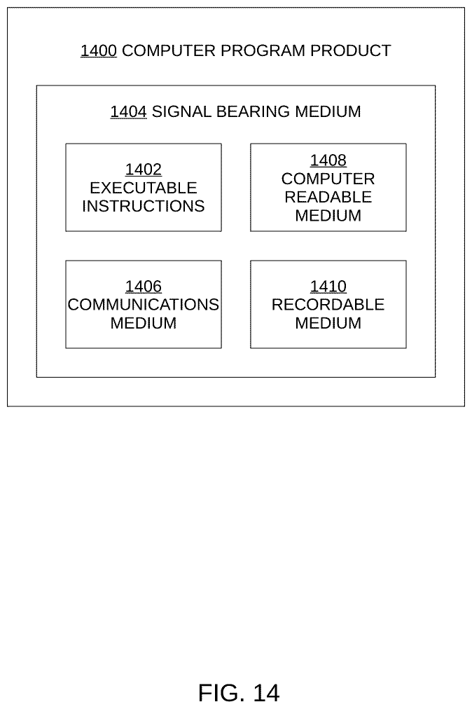

FIG. 14 is a block diagram of an illustrative embodiment of a computer program product 1400 for implementing methods for imaging a region of patient anatomy for a radiation therapy system that includes a rotatable gantry operable to rotate about a treatment isocenter of the radiation therapy system, according to various embodiments. Computer program product 1400 may include a signal bearing medium 1404. Signal bearing medium 1404 may include one or more sets of executable instructions 1402 that, when executed by, for example, a processor of a computing device, may provide at least the functionality described above with respect to FIGS. 1-13.

In some implementations, signal bearing medium 1404 may encompass a non-transitory computer readable medium 1408, such as, but not limited to, a hard disk drive, a Compact Disc (CD), a Digital Video Disk (DVD), a digital tape, memory, etc. In some implementations, signal bearing medium 1404 may encompass a recordable medium 1410, such as, but not limited to, memory, read/write (R/W) CDs, R/W DVDs, etc. In some implementations, signal bearing medium 1404 may encompass a communications medium 1406, such as, but not limited to, a digital and/or an analog communication medium (e.g., a fiber optic cable, a waveguide, a wired communications link, a wireless communication link, etc.). Computer program product 1400 may be recorded on non-transitory computer readable medium 1408 or another similar recordable medium 1410.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method, or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable processors or gate arrays.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

### EXAMPLE CLAUSES

Further aspects of these teachings are provided by the subject matter of the following clauses (where it will be understood that any of these clauses can be combined with one or more of the other clauses as appropriate). Depending on the desired implementation, clause 2 may be combined with clause 1; clause 3 with clause 1 and/or clause 2; clause 4 with one or more of clauses 1-3; clause 5 with one or more of clauses 1-4; clause 6 with one or more of clauses 1-5, clause 7 with one or more of clauses 1-6; clause 8 with one or more of causes 1-7; clause 9 with one or more of clauses 1-8, clause 10 with one or more of clauses 1-9; clause 11 with one or more of clauses 1-10; clause 12 with one or more of clauses 1-11; clause 13 with one or more of clauses 1-12; clause 14 with one or more of clauses 1-13; and clause 15 with one or more of clauses 1-14. This also applies to clause 16, which may be combined with one or more of clauses 17-21. This also applies to clause 22, which may be combined with one or more of clauses 2-15. This also applies to clause 23, which may be combined with one or more of clauses 24-28. This also applies to clause 29, which may be combined with one or more of clauses 17-21.

Clause 1. A radiation therapy system, comprising: a rotatable gantry operable to rotate about a treatment isocenter of the radiation therapy system; a treatment radiation source mounted on the rotatable gantry; a first x-ray imaging system mounted on the rotatable gantry; a second x-ray imaging system that is fixed in position while the rotatable gantry rotates about the treatment isocenter; and a controller, wherein the controller performs the steps of: causing the first x-ray imaging system to generate first image information for a region surrounding the treatment isocenter; causing the second x-ray imaging system to generate second image information for the region surrounding the treatment isocenter; and receiving the first image information from the first x-ray imaging system and the second image information from the second x-ray imaging system.

Clause 2. The radiation therapy system of clause 1, wherein the second x-ray imaging system is mounted on a couch of the radiation therapy system.

Clause 3. The radiation therapy system of clause 1 or clause 2, wherein an x-ray detector of the second x-ray imaging system is positioned above the couch and an x-ray source of the second x-ray imaging system is positioned below the couch.

Clause 4. The radiation therapy system of one or more of clauses 1-3, wherein an x-ray detector of the second x-ray imaging system is positioned below the couch and an x-ray source of the second x-ray imaging system is positioned above the couch.

Clause 5. The radiation therapy system of one or more of clauses 1-4, wherein each of an x-ray detector of the second x-ray imaging system and an x-ray source of the second x-ray imaging system is positioned in a plane that is perpendicular to a support surface of the couch and parallel to a longitudinal axis of the couch. [supplemental imaging system is aligned with the couch so the patient treatment region is visible at every couch angle

Clause 6. The radiation therapy system of one or more of clauses 1-5, wherein the second x-ray imaging system is mounted on a couch of the radiation therapy system via an interchangeable arm.

Clause 7. The radiation therapy system of one or more of clauses 1-6, wherein the second x-ray imaging system is mounted on a couch of the radiation therapy system via an adjustable arm.

Clause 8. The radiation therapy system of one or more of clauses 1-7, wherein the adjustable arm comprises a robotic arm that is controlled by the controller.

Clause 9. The radiation therapy system of one or more of clauses 1-8, wherein a line of sight between an x-ray detector of the second x-ray imaging system and an x-ray source of the second x-ray imaging system is outside of a region swept by one or more components mounted on the rotatable gantry when the rotatable gantry rotates about the treatment isocenter.

Clause 10. The radiation therapy system of one or more of clauses 1-9, wherein at least one component of the second x-ray imaging system is mounted on a surface external to the rotatable gantry or couch of the radiation therapy system.

Clause 11. The radiation therapy system of one or more of clauses 1-10, wherein the at least one component of the second x-ray imaging system comprises an x-ray detector and the surface external to the rotatable gantry comprises one of a first location on a floor of a treatment room containing the radiation therapy system and a second location on a ceiling of the treatment room.

Clause 12. The radiation therapy system of one or more of clauses 1-11, wherein the at least one component of the second x-ray imaging system comprises an x-ray source and the surface external to the rotatable gantry comprises one of a first location on a floor of a treatment room containing the radiation therapy system and a second location on a ceiling of the treatment room.

Clause 13. The radiation therapy system of one or more of clauses 1-12, wherein at least one an x-ray detector or an x-ray source of the second x-ray imaging system is mounted on a robotic arm that is controlled by the controller.

Clause 14. The radiation therapy system of one or more of clauses 1-13, wherein the x-ray detector is mounted on a first robotic arm and the x-ray source is mounted on a second robotic arm.

Clause 15. The radiation therapy system of one or more of clauses 1-14, wherein the rotatable gantry comprises one of an O-ring gantry or a C-arm gantry.

Clause 16. One or more non-transitory computer-readable media storing instructions that, when executed by one or more processors, cause the one or more processors to perform the steps of: causing a first x-ray imaging system mounted on a rotatable gantry operable to rotate about a treatment isocenter of a radiation therapy system to generate first image information for a region of patient anatomy; causing a second x-ray imaging system of the radiation therapy system to generate second image information for the region of patient anatomy, wherein the second x-ray imaging system is fixed in position while the rotatable gantry rotates about the treatment isocenter; receiving the first image information from the first x-ray imaging system and the second image information from the second x-ray imaging system; and generating a first image of the region of patient anatomy based on the first image information and a second image of the region of patient anatomy based on the second image information.

Clause 17. The one or more non-transitory computer-readable media of clause 16, wherein the first x-ray imaging system generates the first image information synchronously with the second x-ray imaging system generating the second image information.

Clause 18. The one or more non-transitory computer-readable media of clause 16 or 17, wherein the region of patient anatomy comprises a region that surrounds the treatment isocenter of the radiation therapy system.

Clause 19. The one or more non-transitory computer-readable media of one or more of clauses 16-18, wherein the steps further include, based on imaging chain information associated with the first x-ray imaging system and imaging chain information associated with the second x-ray imaging system, performing three-dimensional location matching of at least one feature that is included in the first image and in the second image.

Clause 20. The one or more non-transitory computer-readable media of one or more of clauses 16-19, wherein the steps further include, based on the first image and the second image, determining a position in three-dimensional space of the region of patient anatomy relative to the radiation therapy system.

Clause 21. The one or more non-transitory computer-readable media of one or more of clauses 16-20, wherein the steps further include optimizing a patient motion model based on the first image and the second image.

Clause 22. A radiation therapy system, comprising: a rotatable gantry operable to rotate about a treatment isocenter of the radiation therapy system; a treatment radiation source mounted on the rotatable gantry; a first x-ray imaging system mounted on the rotatable gantry; a second x-ray imaging system that is fixed in position while the rotatable gantry rotates about the treatment isocenter; and a controller, wherein the controller performs the steps of: causing the second x-ray imaging system to generate second image information for the region surrounding the treatment isocenter; and receiving the second image information from the second x-ray imaging system.

Clause 23. A computer program storing instructions that, when executed by one or more processors, cause the one or more processors to perform the steps of: causing a first x-ray imaging system mounted on a rotatable gantry operable to rotate about a treatment isocenter of a radiation therapy system to generate first image information for a region of patient anatomy; causing a second x-ray imaging system of the radiation therapy system to generate second image information for the region of patient anatomy, wherein the second x-ray imaging system is fixed in position while the rotatable gantry rotates about the treatment isocenter; receiving the first image information from the first x-ray imaging system and the second image information from the second x-ray imaging system; and generating a first image of the region of patient anatomy based on the first image information and a second image of the region of patient anatomy based on the second image information.

Clause 24. The one or more non-transitory computer-readable media of clause 23, wherein the first x-ray imaging system generates the first image information synchronously with the second x-ray imaging system generating the second image information.

Clause 25. The one or more non-transitory computer-readable media of clause 23 or 24, wherein the region of patient anatomy comprises a region that surrounds the treatment isocenter of the radiation therapy system.

Clause 26. The one or more non-transitory computer-readable media of one or more of clauses 23-25, wherein the steps further include, based on imaging chain information associated with the first x-ray imaging system and imaging chain information associated with the second x-ray imaging system, performing three-dimensional location matching of at least one feature that is included in the first image and in the second image.

Clause 27. The one or more non-transitory computer-readable media of one or more of clauses 23-26, wherein the steps further include, based on the first image and the second image, determining a position in three-dimensional space of the region of patient anatomy relative to the radiation therapy system.

Clause 28. The one or more non-transitory computer-readable media of one or more of clauses 23-27, wherein the steps further include optimizing a patient motion model based on the first image and the second image.

Clause 29. A computer-implemented method of imaging a region of patient anatomy for a radiation therapy system that includes a rotatable gantry operable to rotate about a treatment isocenter of the radiation therapy system, the method comprising: causing a first x-ray imaging system mounted on the rotatable gantry to generate first image information for the region of patient anatomy; causing a second x-ray imaging system of the radiation therapy system to generate second image information for the region of patient anatomy, wherein the second x-ray imaging system is fixed in position while the rotatable gantry rotates about the treatment isocenter; receiving the first image information from the first x-ray imaging system and the second image information from the second x-ray imaging system; and generating a first image of the region of patient anatomy based on the first image information and a second image of the region of patient anatomy based on the second image information.

Clause 30. A computer-implemented method of imaging a region of patient anatomy for a radiation therapy system that includes a rotatable gantry operable to rotate about a treatment isocenter of the radiation therapy system, the method comprising: causing a first x-ray imaging system mounted on the rotatable gantry to generate first image information for the region of patient anatomy; causing a second x-ray imaging system of the radiation therapy system to generate second image information for the region of patient anatomy, wherein the second x-ray imaging system is fixed in position while the rotatable gantry rotates about the treatment isocenter; receiving the first image information from the first x-ray imaging system and the second image information from the second x-ray imaging system; and generating a first image of the region of patient anatomy based on the first image information and a second image of the region of patient anatomy based on the second image information.

Clause 31. The computer-implemented method of clause 30, wherein the first x-ray imaging system generates the first image information synchronously with the second x-ray imaging system generating the second image information.

Clause 32. The computer-implemented method of clause 30 or clause 31, wherein the region of patient anatomy comprises a region that surrounds the treatment isocenter of the radiation therapy system.

Clause 33. The computer-implemented method of one or more of clauses 30-32, further comprising, based on imaging chain information associated with the first x-ray imaging system and imaging chain information associated with the second x-ray imaging system, performing three-dimensional location matching of at least one feature that is included in the first image and in the second image.

Clause 34. The computer-implemented method of one or more of clauses 30-33, further comprising, based on the first image and the second image, determining a position in three-dimensional space of the region of patient anatomy relative to the radiation therapy system.

Clause 35. The computer-implemented method of one or more of clauses 30-34, further comprising, optimizing a patient motion model based on the first image and the second image.

Clause 36. One or more non-transitory computer-readable media storing instructions that, when executed by one or more processors, cause the one or more processors to perform the method of any one of clauses 30-35.

Clause 37. A computer program storing instructions that, when executed by one or more processors, cause the one or more processors to perform the method of any one of clauses 30-35.

While various aspects and embodiments have been disclosed herein, other aspects and embodiments will be apparent to those skilled in the art without departing from the scope and spirit of the described embodiments. The various aspects and embodiments disclosed herein are for purposes of illustration and are not intended to be limiting, with the true scope and spirit being indicated by the following claims.

1. A radiation therapy system, comprising:
a rotatable gantry operable to rotate about a treatment isocenter of the radiation therapy system;
a treatment radiation source mounted on the rotatable gantry;
a first x-ray imaging system mounted on the rotatable gantry;
a second x-ray imaging system that is fixed in position while the rotatable gantry rotates about the treatment isocenter; and
a controller, wherein the controller performs the steps of:
causing the first x-ray imaging system to generate first image information for a region surrounding the treatment isocenter;
causing the second x-ray imaging system to generate second image information for the region surrounding the treatment isocenter; and
receiving the first image information from the first x-ray imaging system and the second image information from the second x-ray imaging system.

2. The radiation therapy system of claim 1, wherein the second x-ray imaging system is mounted on a couch of the radiation therapy system.

3. The radiation therapy system of claim 2, wherein an x-ray detector of the second x-ray imaging system is positioned above the couch and an x-ray source of the second x-ray imaging system is positioned below the couch.

4. The radiation therapy system of claim 2, wherein an x-ray detector of the second x-ray imaging system is positioned below the couch and an x-ray source of the second x-ray imaging system is positioned above the couch.

5. The radiation therapy system of claim 2, wherein each of an x-ray detector of the second x-ray imaging system and an x-ray source of the second x-ray imaging system is positioned in a plane that is perpendicular to a support surface of the couch and parallel to a longitudinal axis of the couch.

6. The radiation therapy system of claim 1, wherein the second x-ray imaging system is mounted on a couch of the radiation therapy system via an interchangeable arm.

7. The radiation therapy system of claim 1, wherein the second x-ray imaging system is mounted on a couch of the radiation therapy system via an adjustable arm.

8. The radiation therapy system of claim 7, wherein the adjustable arm comprises a robotic arm that is controlled by the controller.

9. The radiation therapy system of claim 1, wherein a line of sight between an x-ray detector of the second x-ray imaging system and an x-ray source of the second x-ray imaging system is outside of a region swept by one or more components mounted on the rotatable gantry when the rotatable gantry rotates about the treatment isocenter.

10. The radiation therapy system of claim 1, wherein at least one component of the second x-ray imaging system is mounted on a surface external to the rotatable gantry or couch of the radiation therapy system.

11. The radiation therapy system of claim 10, wherein the at least one component of the second x-ray imaging system comprises an x-ray detector and the surface external to the rotatable gantry comprises one of a first location on a floor of a treatment room containing the radiation therapy system and a second location on a ceiling of the treatment room.

12. The radiation therapy system of claim 10, wherein the at least one component of the second x-ray imaging system comprises an x-ray source and the surface external to the rotatable gantry comprises one of a first location on a floor of a treatment room containing the radiation therapy system and a second location on a ceiling of the treatment room.

13. The radiation therapy system of claim 1, wherein at least one an x-ray detector or an x-ray source of the second x-ray imaging system is mounted on a robotic arm that is controlled by the controller.

14. The radiation therapy system of claim 13, wherein the x-ray detector is mounted on a first robotic arm and the x-ray source is mounted on a second robotic arm.

15. The radiation therapy system of claim 1, wherein the rotatable gantry comprises one of an O-ring gantry or a C-arm gantry.

16. One or more non-transitory computer-readable media storing instructions that, when executed by one or more processors, cause the one or more processors to perform the steps of:
causing a first x-ray imaging system mounted on a rotatable gantry operable to rotate about a treatment isocenter of a radiation therapy system to generate first image information for a region of patient anatomy;
causing a second x-ray imaging system of the radiation therapy system to generate second image information for the region of patient anatomy, wherein the second x-ray imaging system is fixed in position while the rotatable gantry rotates about the treatment isocenter;
receiving the first image information from the first x-ray imaging system and the second image information from the second x-ray imaging system; and
generating a first image of the region of patient anatomy based on the first image information and a second image of the region of patient anatomy based on the second image information.

17. The one or more non-transitory computer-readable media of claim 16, wherein the first x-ray imaging system generates the first image information synchronously with the second x-ray imaging system generating the second image information.

18. The one or more non-transitory computer-readable media of claim 16, wherein the region of patient anatomy comprises a region that surrounds the treatment isocenter of the radiation therapy system.

19. The one or more non-transitory computer-readable media of claim 16, wherein the steps further include, based on imaging chain information associated with the first x-ray imaging system and imaging chain information associated with the second x-ray imaging system, performing three-dimensional location matching of at least one feature that is included in the first image and in the second image.

20. The one or more non-transitory computer-readable media of claim 16, wherein the steps further include, based on the first image and the second image, determining a position in three-dimensional space of the region of patient anatomy relative to the radiation therapy system.

21. The one or more non-transitory computer-readable media of claim 16, wherein the steps further include optimizing a patient motion model based on the first image and the second image.

22. A radiation therapy system, comprising:
a rotatable gantry operable to rotate about a treatment isocenter of the radiation therapy system;
a treatment radiation source mounted on the rotatable gantry;
a first x-ray imaging system mounted on the rotatable gantry;
a second x-ray imaging system that is fixed in position while the rotatable gantry rotates about the treatment isocenter; and
a controller, wherein the controller performs the steps of:
causing the second x-ray imaging system to generate second image information for the region surrounding the treatment isocenter; and
receiving the second image information from the second x-ray imaging system.
